(19) United States
(12) Patent Application Publication
(10) Pub. No.: US 2013/0005797 A1
(43) Pub. Date: Jan. 3, 2013

(54) ENDOSOMOLYTIC POLY(AMIDOAMINE) DISULFIDE POLYMERS FOR THE DELIVERY OF OLIGONUCLEOTIDES

(75) Inventors: Marina Busuek, Center Valley, PA (US); Steven L. Colletti, Princeton Junction, NJ (US); Rubina G. Parmar, Harleysville, PA (US); Weimin Wang, Southampton, PA (US); J. Michael Williams, Hillsborough, NJ (US)

(73) Assignee: Merck Sharp & Dohme Corp.

(21) Appl. No.: 13/634,579

(22) PCT Filed: Mar. 14, 2011
(86) PCT No.: PCT/US11/28260

§ 371 Date: Sep. 13, 2012

Related U.S. Application Data
(60) Provisional application No. 61/315,223, filed on Mar. 18, 2010.

(51) Int. Cl. C08G 69/42; A61K 48/00

(52) U.S. Cl. 514/44 R; 528/337

(57) ABSTRACT

The present invention provides endosomolytic poly(amidoamine)disulfide polymers, polyconjugates, compositions and methods for the delivery of oligonucleotides for therapeutic purposes.

### BACKGROUND OF THE INVENTION

Oligonucleotides conjugated to polymers are known. Further, the delivery of oligonucleotides conjugated to polymers (polyconjugates) for therapeutic purposes is also known. See WO2000/34343; WO2008/022309; and Rozema et al. PNAS (2008) 104, 32: 12982-12987.

Poly(amidoamine) disulfide polymers are known. Ou et al. Bioconjugate Chem. (2008) 19: 626-633 and Lin et al. J. of Controlled Release (2008) 126: 166-174.

Polyconjugates have numerous toxicities associated with extensive circulation half-life. It is thus an object of the invention to provide polyconjugates that are biodegradable. Herein, we disclose and describe novel endosomolytic poly(amidoamine) disulfide polymers and polyconjugates useful for the delivery of oligonucleotides for therapeutic purposes. The poly(amidoamine) disulfide polymers of the instant invention are novel and contain both cationic and aliphatic functional moieties. These polymers are biodegradable and are therefore less toxic.

### SUMMARY OF THE INVENTION

The present invention provides endosomolytic poly(amidoamine) disulfide polymers, polyconjugates, compositions and methods for the delivery of oligonucleotides for therapeutic purposes.

### BRIEF DESCRIPTION OF THE FIGURES

FIG. 1. RBC Hemolysis Data of Polymers at pH 5.5.

FIG. 2. RBC Hemolysis Data of Masked Polyconjugate.

FIG. 3. In-Vitro Data.

FIG. 4. In-Vivo Data.

### DETAILED DESCRIPTION OF THE INVENTION

An embodiment of the instant invention is a polymer of Formula Z:

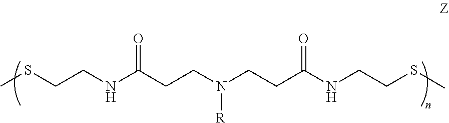

wherein:

n is 2 to 250;

R is independently selected from A, B and C, wherein at least two of A, B and C are present in the polymer and the polymer must include at least one cationic component (A or B) and at least one aliphatic component (B or C);

A is a first cationic component;

B is a second cationic component or a second aliphatic component; and

C is a first aliphatic component; or stereoisomer thereof.

In a second embodiment of the instant invention is a polymer of Formula Z:

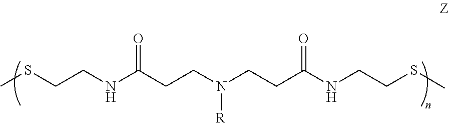

wherein:

n is 2 to 250;

R is independently selected from A, B and C, wherein at least two of A, B and C are present in the polymer and the polymer must include at least one cationic component (A or B) and at least one aliphatic component (B or C);

A is a first cationic component selected from an amine (primary, secondary, tertiary or quaternary), a nitrogen heterocycle, an aldimine, a hydrazide and a hydrazone;

B is a second cationic component selected from an amine (primary, secondary, tertiary or quaternary), a nitrogen heterocycle, an aldimine, a hydrazide and a hydrazone, or a second aliphatic component; and

C is a first aliphatic component;

or stereoisomer thereof.

In a third embodiment of the instant invention is a polymer of Formula Z:

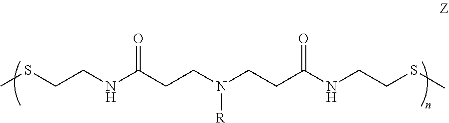

wherein:

n is 2 to 250;

R is independently selected from A, B and C, wherein A, B and C are all present in the polymer;

A is 2-(2-aminoethoxy)ethyl;

B is 2-(1H-imidazol-4-yl)ethyl; and

C is dodecyl;

or a stereoisomer thereof.

In another embodiment of the polymer of Formula Z, R is independently selected from A, B and C, wherein A, B and C are all present in the polymer and wherein A, B and C are different from one another.

In another embodiment of the polymer of Formula Z, R is independently selected from A, B and C, wherein A, B and C are all present in the polymer and wherein A and B are different from one another.

In another embodiment of the polymer of Formula Z, R is independently selected from A, B and C, wherein A, B and C are all present in the polymer and wherein B and C are different from one another.

In another embodiment of the polymer of Formula Z, substituent A must comprise at least 50% of the polymer.

In another embodiment of the polymer of Formula Z, substituent B must comprise at least 50% of the polymer.

In another embodiment of the polymer of Formula Z, substituent C must comprise at least 50% of the polymer.

Another aspect of the instant invention is a composition comprising a polymer of Formula Z and an oligonucleotide.

Another aspect of the instant invention is a composition comprising a polymer of Formula Z, a masking agent and an oligonucleotide.

Another aspect of the instant invention is a composition comprising a polymer of Formula Z, a masking agent, a targeting agent and an oligonucleotide.

Another aspect of the instant invention is a method of treating a disease in a patient by administering a composition of the instant invention.

### DEFINITIONS

“Cationic component” means a chemical moiety that carries a positive charge. A cationic component includes amines, nitrogen heterocycles, aldimines, hydrazides and hydrazones. Cationic component is further defined as a “first cationic component” and a “second cationic component”. This further delineation is meant to show that the first cationic component is different than the second cationic component.

“Amine (primary, secondary, tertiary or quaternary)” means organic compounds and functional groups that contain a basic nitrogen atom with a lone pair. Amines are derivatives of ammonia, wherein one or more hydrogen atoms have been replaced by a substituent such as an alkyl or aryl group.

“Nitrogen heterocycle” means an organic compound containing at least one atom of carbon and at least one atom on nitrogen within a ring structure. These structures may comprise either simple aromatic rings or non-aromatic rings.

“Aldimine” means a class of organic compounds with the general formula R—CH═N—R′.

“Hydrazide” means a class of organic compounds sharing a common functional group characterized by a nitrogen to nitrogen covalent bond with 4 substituents with at least one of them being an acyl group. The general structure for a hydrazide is (R₁═O)R₂—N—N—R₃R₄.

“Hydrazone” means a class of organic compounds with the structure R₁R₂C═NNH₂.

“Aliphatic component” means a compound composed of carbon and hydrogen. Aliphatic compounds can be cyclic, like cyclohexane, or acyclic, like hexane. Aliphatic compounds can be saturated, like hexane, or unsaturated, like hexene. Aliphatic compounds can be straight chains, branched chains, or non-aromatic rings (in which case they are called alicyclic). Aliphatic compounds can be joined by single bonds (alkanes), double bonds (alkenes), or triple bonds (alkynes). An aliphatic component includes aromatic components and steriods. Aliphatic component is further defined as a “first aliphatic component” and a “second aliphatic component”. This further delineation is meant to show that the first aliphatic component is different than the second aliphatic component.

“Aromatic component” means a compound composed of hydrocarbon with a conjugated cyclic molecular structure.

“Steroid” includes, for example, cholesterol.

“Masking agent” means a molecule which, when linked to a polymer, shields, inhibits or inactivates one or more properties (biophysical or biochemical characteristics) of the polymer. See WO20081022309 for a more detailed description of masking agents.

“Targeting agent” means an agent that can deliver a polymer or polyconjugate to target cells or tissues, or specific cells types. Targeting agents enhance the association of molecules with a target cell. Thus, targeting agents can enhance the pharmacokinetic or biodistribution properties of a polyconjugate to which they are attached to improve cellular distribution and cellular uptake of the conjugate. See WO2008/022309 for a more detailed description of targeting agents.

“Polymer” means a molecule built up by repetitive bonding together of smaller units called monomers. A polymer can be linear, branched network, star, comb, or ladder type. A polymer can be a homopolymer in which a single monomer is used or a polymer can be copolymer in which two or more different monomers are used. Copolymers may by alternating, random (statistical), block and graft (comb). The monomers in random copolymers have no definite order or arrangement along any given chain. The general compositions of such polymers are reflective of the ratio of input monomers. However, the exact ratio of one monomer to another may differ between chains. The distribution of monomers may also differ along the length of a single polymer. Also, the chemical properties of a monomer may affect its rate of incorporation into a random copolymer and its distribution within the polymer. Thus, while the ratio of monomers in a random polymer is dependent on the input ratio of monomer, the input ratio may not match exactly the ratio of incorporated monomers. See WO2008/022309 for a more detailed description of polymers.

“Oligonucleotide” means deoxyribonucleic acid (DNA) and ribonucleic acid (RNA) and combinations of DNA, RNA and other natural and synthetic nucleotides. DNA maybe in form of cDNA, in vitro polymerized DNA, plasmid DNA, parts of a plasmid DNA, genetic material derived from a virus, linear DNA, vectors (P1, PAC, BAC, YAC, and artificial chromosomes), expression vectors, expression cassettes, chimeric sequences, recombinant DNA, chromosomal DNA, anti-sense DNA, or derivatives of these groups. RNA may be in the form of messengerRNA (mRNA), in vitro polymerized RNA, recombinant RNA, transfer RNA (tRNA), small nuclear RNA (snRNA), ribosomal RNA (rRNA), chimeric sequences, anti-sense RNA, interfering RNA, small interfering RNA (siRNA), microRNA (miRNA), ribozymes, external guide sequences, small non-messenger RNAs (snmRNA), untranslatedRNA (utRNA), snoRNAs (24-mers, modified snmRNA that act by an anti-sense mechanism), tiny non-coding RNAs (tncRNAs), small hairpin RNA (shRNA), or derivatives of these groups. In addition, DNA and RNA may be single, double, triple, or quadruple stranded. Double, triple, and quadruple stranded polynucleotide may contain both RNA and DNA or other combinations of natural and/or synthetic nucleic acids. Oligonucleotides can be chemically modified. The use of chemically modified oligonucleotides can improve various properties of the oligonucleotides including, but not limited to: resistance to nuclease degradation in vivo, cellular uptake, activity, and sequence-specific hybridization. Non-limiting examples of such chemical modifications include: phosphorothioate internucleotide linkages, LNA, 2′-O-methyl ribonucleotides, 2′-deoxy-2′-fluoro ribonucleotides, 2′-deoxy ribonucleotides, “universal base” nucleotides, 5-C-methyl nucleotides, and inverted deoxyabasic residue incorporation. These chemical modifications, when used in various oligonucleotide constructs, are shown to preserve oligonucleotide activity in cells while at the same time, dramatically increasing the serum stability of these compounds. Chemically modified siRNA can also minimize the possibility of activating interferon activity in humans. See WO2008/022309 for a more detailed description of oligonucleotides.

“Patient” means a mammal, typically a human, in need of treatment for a disease.

“Disease” means a disorder or incorrectly functioning organ, part, structure, or system of the body resulting from the effect of genetic or developmental errors, infection, poisons, nutritional deficiency or imbalance, toxicity, or unfavorable environmental factors; illness; sickness; ailment.

In an embodiment, a cationic component is selected from an amine (primary, secondary, tertiary or quaternary), a nitrogen heterocycle, an aldimine, a hydrazide and a hydrazone. In another embodiment, a cationic component is selected from, 2-(2-aminoethoxy)ethyl, 2-(1H-imidazol-4-yl)ethyl, 2-[2-(2-aminoethoxy)ethoxy]ethyl, 3-amino-2-hydroxypropyl, 2-aminoethyl, 4-aminobutyl, 6-aminohexyl, 8-aminooctyl and 10-aminodecyl. In another embodiment, a cationic component is selected 2-(2-aminoethoxy)ethyl and 2-(1H-imidazol-4-yl)ethyl.

In an embodiment, an aliphatic (hydrophobic) component is selected from steroids, an alkyl group, an alkenyl group and an alkynyl group, all of which may be branched or cyclic or acyclic or aromatic. In another embodiment, an aliphatic component is selected from methyl, ethyl, propyl, butyl, hexyl, heptyl, octyl, nonyl, decyl, undecyl, dodecyl, tridecyl, tetradecyl, pentadecyl, hexadecyl, heptadecyl, octadecyl, cholesterol, lipid chains and benzyl. In another embodiment, an aliphatic component is selected from dodecyl, octyl and octadecyl.

In an embodiment, a masking agent is selected from a disubstituted maleic anhydride derivative

In an embodiment, a targeting agent is selected from compounds with affinity to cell surface molecules, cell receptor ligands, and antibodies, antibody fragments, and antibody mimics with affinity to cell surface molecules. In another embodiment, a targeting agent is selected from carbohydrates, glycans, saccharides (including, but not limited to: galactose, galactose derivatives, mannose, and mannose derivatives), vitamins, folate, biotin, aptamers, and peptides (including, but not limited to: ROD-containing peptides, insulin, EGF, and transferrin).

In another embodiment, a targeting agent is selected from N-acetylgalactosamine, mannose and glucose.

In an embodiment, an oligonucleotide is selected from siRNA, miRNA and antisense. In another embodiment, an oligonucleotide is an siRNA.

In an embodiment of Formula Z, n is 2 to 250.

In another embodiment of Formula Z, n is 2 to 225.

In another embodiment of Formula Z, n is 2 to 200.

In another embodiment of Formula Z, n is 2 to 175.

In another embodiment of Formula Z, n is 2 to 150.

In another embodiment of Formula Z, n is 2 to 125.

In another embodiment of Formula Z, n is 2 to 100.

In another embodiment of Formula Z, n is 2 to 75.

In another embodiment of Formula Z, n is 2 to 50.

In another embodiment of Formula Z, n is 2 to 25.

In an embodiment of Formula Z, n is 5 to 250.

In another embodiment of Formula Z, n is 5 to 225.

In another embodiment of Formula Z, n is 5 to 200.

In another embodiment of Formula Z, n is 5 to 175.

In another embodiment of Formula Z, n is 5 to 150.

In another embodiment of Formula Z, n is 5 to 125.

In another embodiment of Formula Z, n is 5 to 100.

In another embodiment of Formula Z, n is 5 to 75.

In another embodiment of Formula Z, n is 5 to 50.

In another embodiment of Formula Z, n is 5 to 25.

In another embodiment of Formula Z, n is 10 to 60.

In another embodiment of Formula Z, n is 15 to 55.

In another embodiment of Formula Z, n is 20 to 50.

In another embodiment of Formula Z, n is 25 to 45.

In another embodiment of Formula Z, n is 30 to 40.

In an embodiment of Formula Z, substituents A and B are selected from, 2-(2-aminoethoxy)ethyl, 2-(1H-imidazol-4-yl)ethyl, 2-[2-(2-aminoethoxy)ethoxy]ethyl, 3-amino-2-hydroxypropyl, 2-aminoethyl, 4-aminobutyl, 6-aminohexyl, 8-aminooctyl and 10-aminodecyl.

In an embodiment of Formula Z, substituents B and C are selected from steroids, an alkyl group, an alkenyl group and an alkynyl group, all of which may be branched or cyclic or acyclic or aromatic.

In another embodiment of Formula Z, substituents B and C are selected from methyl, ethyl, propyl, butyl, hexyl, heptyl, octyl, nonyl, decyl, undecyl, dodecyl, tridecyl, tetradecyl, pentadecyl, hexadecyl, heptadecyl, octadecyl, cholesterol, lipid chains and benzyl.

In another embodiment of Formula Z, substituents B and C are selected from methyl, ethyl, propyl, butyl, hexyl, heptyl, octyl, nonyl, decyl, undecyl, dodecyl, tridecyl, tetradecyl, pentadecyl, hexadecyl, heptadecyl, octadecyl and cholesterol.

In another embodiment of Formula Z, substituents B and C are selected from dodecyl, octyl and octadecyl.

In an embodiment of Formula Z, substituent A is 2-(2-aminoethoxy)ethyl.

In an embodiment of Formula Z, substituent B is 2-(1H-imidazol-4-yl)ethyl.

In an embodiment of Formula Z, substituent C is dodecyl.

### Formulation

The polyconjugate (composition of the polymer of Formula Z and an oligonucleotide) is formed by covalently linking the oligonucleotide to the polymer. Conjugation of the oligonucleotide to the polymer can be performed in the presence of an excess of polymer.

Because the oligonucleotide and the polymer may be of opposite charge during conjugation, the presence of excess polymer can reduce or eliminate aggregation of the polyconjugate. Excess polymer can be removed from the polyconjugate prior to administration of the polyconjugate to a patient. Alternatively, excess polymer can be co-administered with the polyconjugate to the patient.

Similarly, the polymer can be conjugated to a masking agent in the presence of an excess of polymer or masking agent. Because the oligonucleotide and the polymer may be of opposite charge during conjugation, the presence of excess polymer can reduce or eliminate aggregation of the polyconjugate. Excess polymer can be removed from the polyconjugate prior to administration of the polyconjugate to a patient. Alternatively, excess polymer can be co-administered with the polyconjugate to the patient. The polymer can be modified prior to or subsequent to conjugation of the oligonucleotide to the polymer.

Parenteral routes of administration include intravascular (intravenous, interarterial), intramuscular, intraparenchymal, intradermal, subdermal, subcutaneous, intratumor, intraperitoneal, intrathecal, subdural, epidural, and intralymphatic injections that use a syringe and a needle or catheter. Intravascular herein means within a tubular structure called a vessel that is connected to a tissue or organ within the body. Within the cavity of the tubular structure, a bodily fluid flows to or from the body part. Examples of bodily fluid include blood, cerebrospinal fluid (CSF), lymphatic fluid, or bile. Examples of vessels include arteries, arterioles, capillaries, venules, sinusoids, veins, lymphatics, bile ducts, and ducts of the salivary or other exocrine glands. The intravascular route includes delivery through the blood vessels such as an artery or a vein. The blood circulatory system provides systemic spread of the pharmaceutical. An administration route involving the mucosal membranes is meant to include nasal, bronchial, inhalation into the lungs, or via the eyes. Intraparenchymal includes direct injection into a tissue such as liver, lung, heart, muscle (skeletal muscle or diaphragm), spleen, pancreas, brain (including intraventricular), spinal cord, ganglion, lymph nodes, adipose tissues, thyroid tissue, adrenal glands, kidneys, prostate, and tumors. Transdermal routes of administration have been affected by patches and iontophoresis. Other epithelial routes include oral, nasal, respiratory, rectum, and vaginal routes of administration.

The polyconjugates can be injected in a pharmaceutically acceptable carrier solution. Pharmaceutically acceptable refers to those properties and/or substances which are acceptable to the patient from a pharmacological/toxicological point of view. The phrase pharmaceutically acceptable refers to molecular entities, compositions, and properties that are physiologically tolerable and do not typically produce an allergic or other untoward or toxic reaction when administered to a patient. Preferably, as used herein, the term pharmaceutically acceptable means approved by a regulatory agency of the Federal or a state government or listed in the U.S. Pharmacopeia or other generally recognized pharmacopeia for use in animals, and more particularly in humans.

### Utility

The polyconjugates (compositions of a polymer of Formula Z and an oligonucleotide) of the instant invention may be used for research purposes or to produce a change in a cell that can be therapeutic. The use of polyconjugates for therapeutic purposes is known. See WO2000/34343; WO2008/022309; and Rozema et al. PNAS (2008) 104, 32: 12982-12987.

### EXAMPLES

Examples and schemes provided are intended to assist in a further understanding of the invention. Particular materials employed, species and conditions are intended to be further illustrative of the invention and not limitative of the reasonable scope thereof.

### General Polymer Synthesis

The monomers were weighed and brought up in 30% ethanol solution in water. The reaction mixture was stirred at 55° C. in dark for 5 days under nitrogen atmosphere. After 5 days, polymerization was quenched by adding 10 mol % excess of amine to consume any unreacted acrylate. After that, polymer was precipitated with diethylether and dried. Polymers with Boc-protected oligoamines were deprotected by TFA. The crude polymer was precipitated again in diethylether. The polymer was further purified by dialysis.

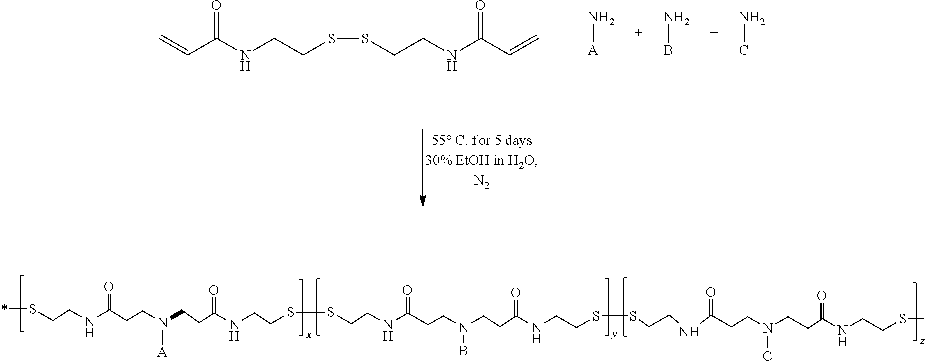

### Polymer Synthesis (Scheme 1)

In a typical experiment, cystaminebisacrylamide (125 mg, 0.48 mmol, 1 equiv), tert-butyl [2-(2-aminoethoxy)ethyl]carbamate: 39 mg, 0.192 mmol, 0.4 equiv), histamine (16 mg, 0.144 mmol, 0.3 equiv) and dodecylamine (27 mg, 0.144 mmol, 0.3 equiv) were weighed in the reaction flask and 1 ml of 30% ethanol solution in water was added to it. The reaction mixture was stirred at 55° C. for 5 days in dark under nitrogen atmosphere. After 5 days, polymerization was quenched by adding 10 mol % excess of dodecylamine to consume any unreacted bisacrylamide group. After that, polymer was precipitated with 100 ml diethylether and dried.

The deprotection of Boc-amine was carried out by dissolving protected polymer in 2 ml of TFA/TIS/H₂O 95/2.5/2.5 solutions for 30 min at room temperature. The crude polymer was precipitated out again in 100 ml diethylether and dried. It was further purified by dialysis using 2k cut off membrane against Milli-Q water and then lyophilized.

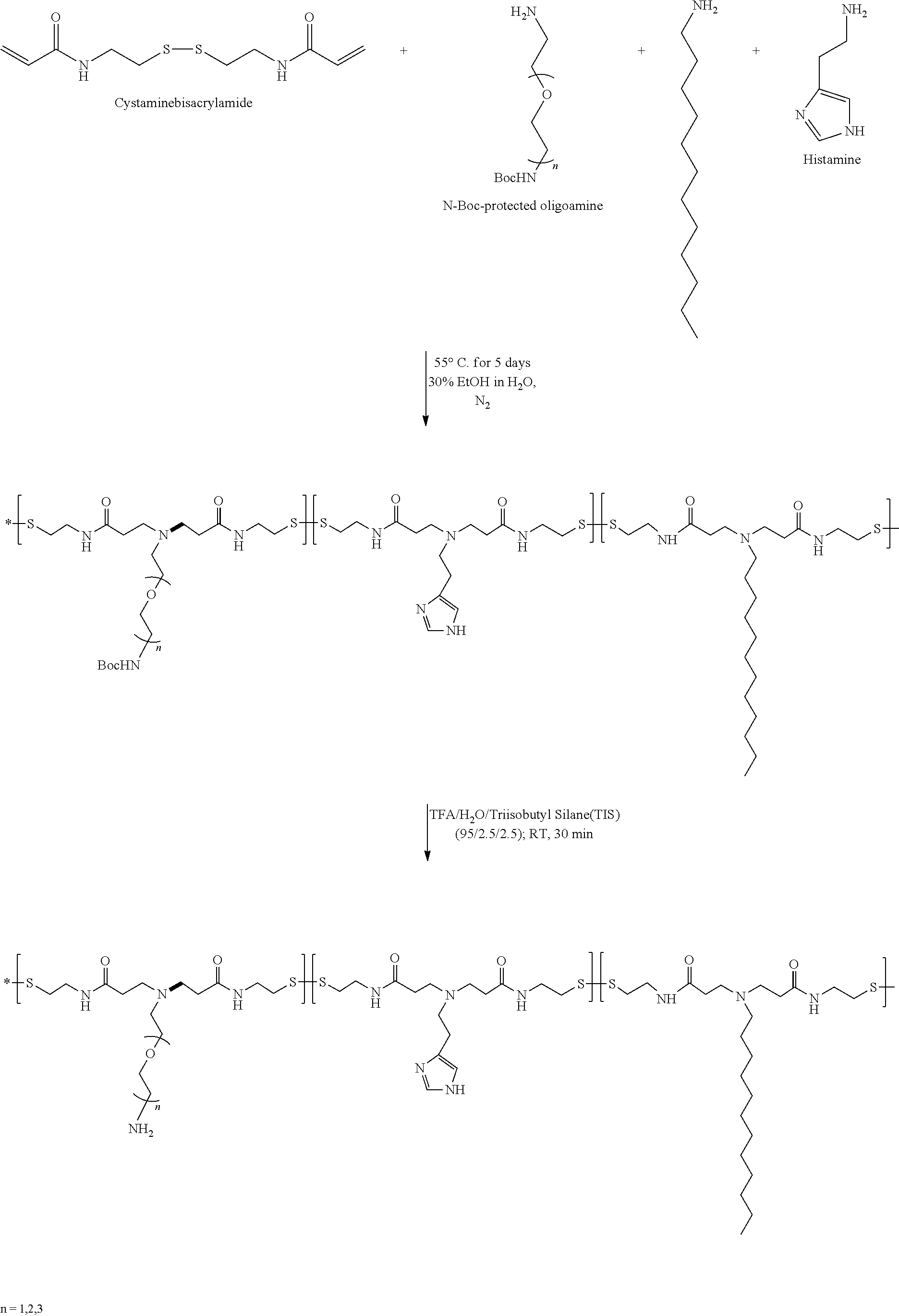

1H and 13C NMR spectra were recorded on Varian spectrometer operating at 500 MHz, 1H NMR spectra were in full accordance with the expected structures. No signals were present in the region between 5 and 7 ppm, corresponding to the acrylamide group, indicating that these polymers have amino-capped end groups. All NMR spectra were taken in deuterated methanol. The molecular weight and polydispersity (Mw/Mn) of the synthesized polymers were determined by GPC relative to polystyrene standards (Sigma-Aldrich) using a Waters 2695, Waters 2414 RI detector and TSK-GEL Alpha-2500 column. Total amine content and the incorporation ratios of monomers in the polymers were calculated by treating the polymers with excess of DTT and then analysed by LC-MS.

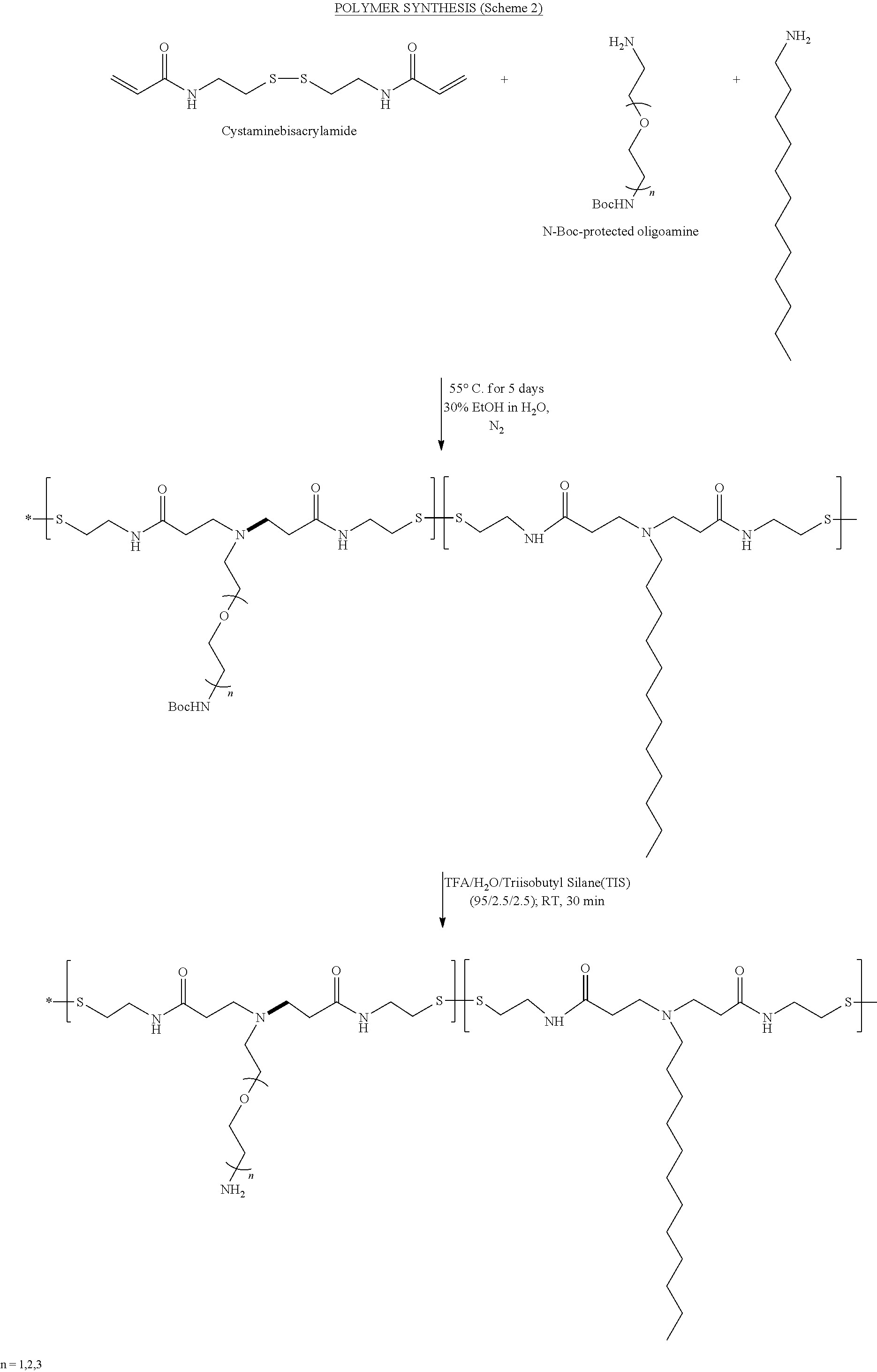

The following polymers were prepared according to the General Reaction Scheme and Schemes above.

### Polymer 1

Polymers of the instant invention include:

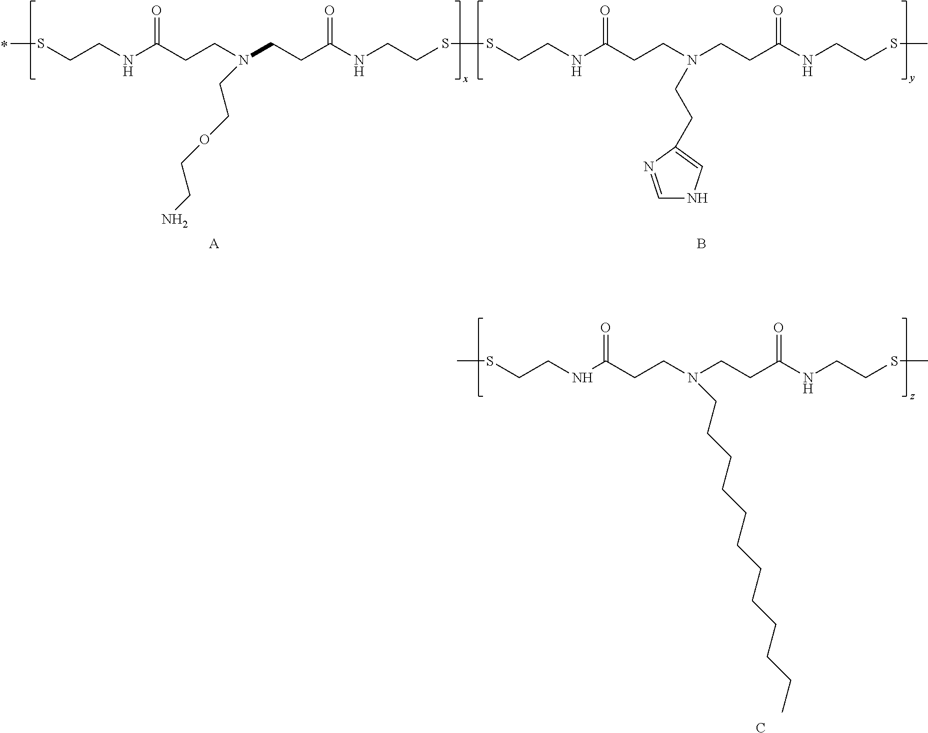

wherein x, y and z are between 0 to 50 and individual polymers in Table 1 include substituents A, B and C in the identified % ratios.

TABLE 1

| A | B | C |
|---|---|---|
| 30 | 50 | 20 |
| 40 | 30 | 30 |
| 30 | 20 | 50 |
| 40 | 50 | 10 |
| 50 | 40 | 10 |
| 30 | 60 | 10 |
| 30 | 30 | 40 |
| 50 | 20 | 30 |
| 60 | 30 | 10 |
| 80 | 0 | 20 |
| 70 | 0 | 30 |
| 50 | 0 | 50 |
| 40 | 0 | 60 |

### Polymer 2

Polymers of the instant invention include:

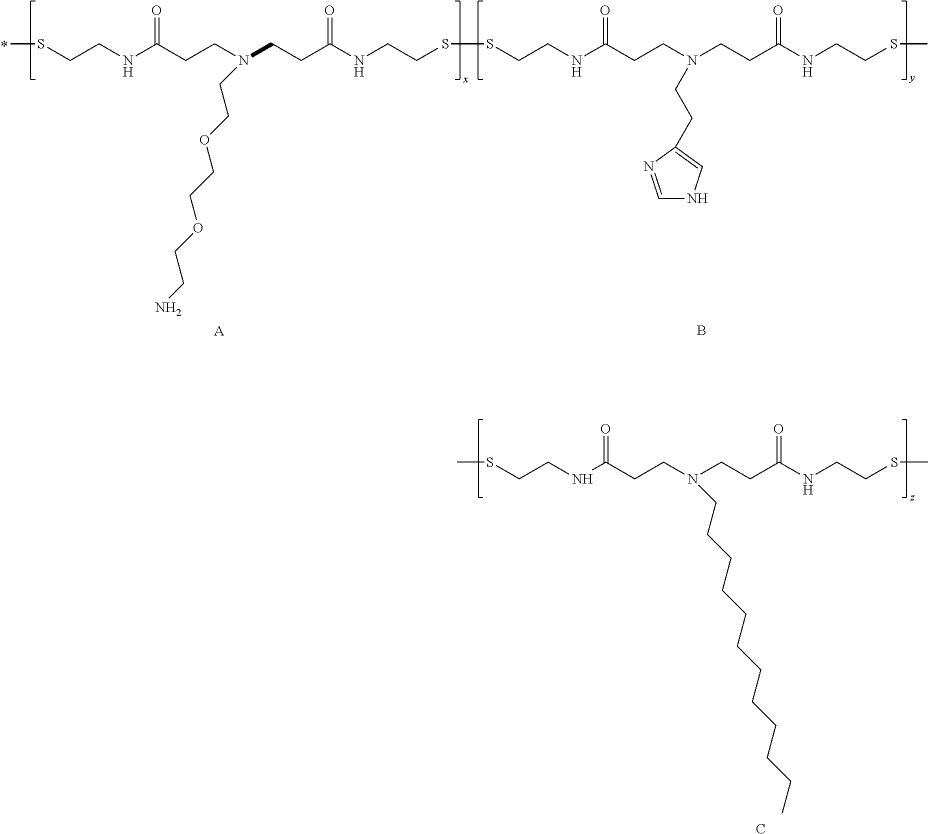

wherein x, y and z are between 0 to 50 and individual polymers in Table 2 include substituents A, B and C in the identified % ratios.

TABLE 2

| A | B | C |
|---|---|---|
| 40 | 30 | 30 |
| 30 | 20 | 50 |
| 50 | 40 | 10 |
| 70 | 0 | 30 |
| 50 | 0 | 50 |

### Polymer 3

Polymers of the instant invention include:

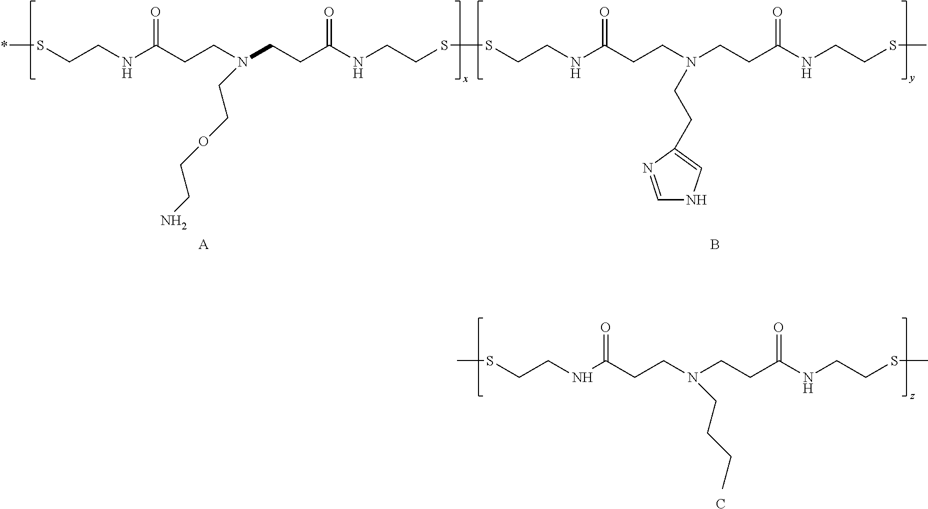

wherein x, y and z are between 0 to 50 and individual polymers in Table 3 include substituents A, B and C in the identified % ratios.

TABLE 3

| A | B | C |
|---|---|---|
| 80 | 0 | 20 |
| 50 | 20 | 30 |
| 70 | 0 | 30 |

### Polymer 4

Polymers of the instant invention include:

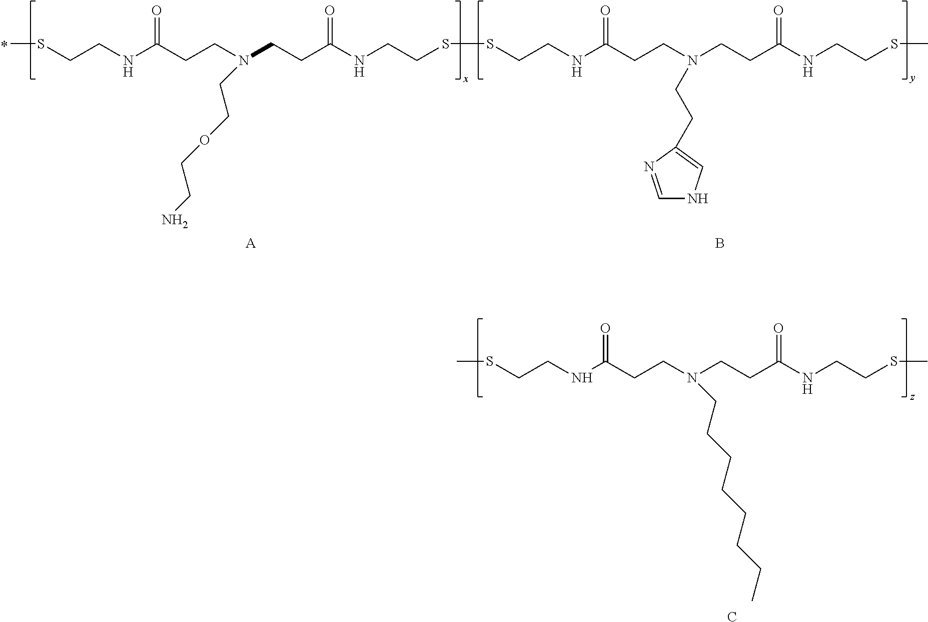

wherein x, y and z are between 0 to 50 and individual polymers in Table 4 include substituents A, B and C in the identified % ratios.

TABLE 4

| A | B | C |
|---|---|---|
| 70 | 0 | 30 |
| 50 | 0 | 50 |
| 40 | 0 | 60 |
| 40 | 20 | 40 |

### Polymer 5

Polymers of the instant invention include:

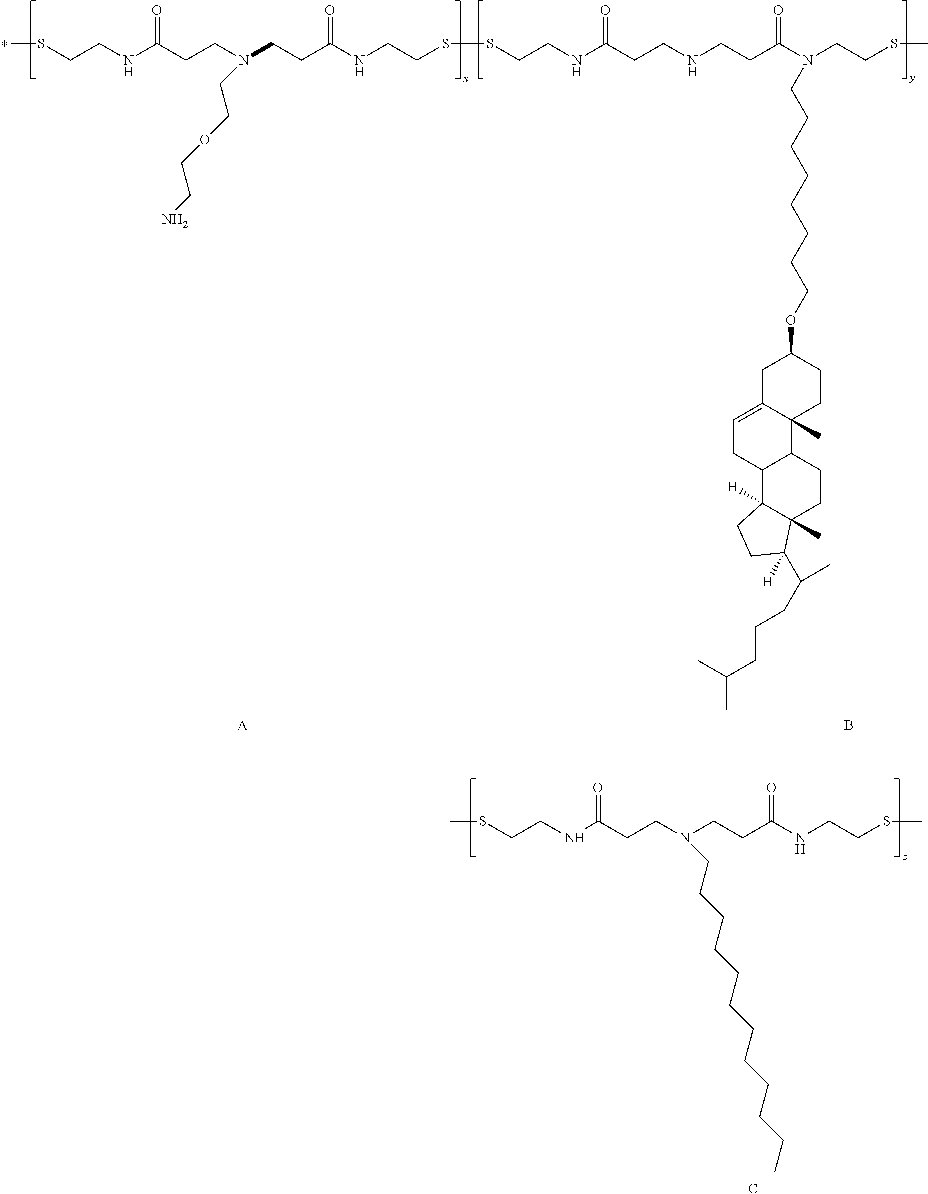

wherein x, y and z are between 0 to 50 and individual polymers in Table 5 include substituents A, B and C in the identified % ratios.

TABLE 5

| A | B | C |
|---|---|---|
| 80 | 20 | 0 |
| 60 | 40 | 0 |
| 70 | 10 | 20 |
| 70 | 20 | 10 |
| 50 | 25 | 25 |
| 50 | 40 | 10 |

### Polymer 6

Polymers of the instant invention include:

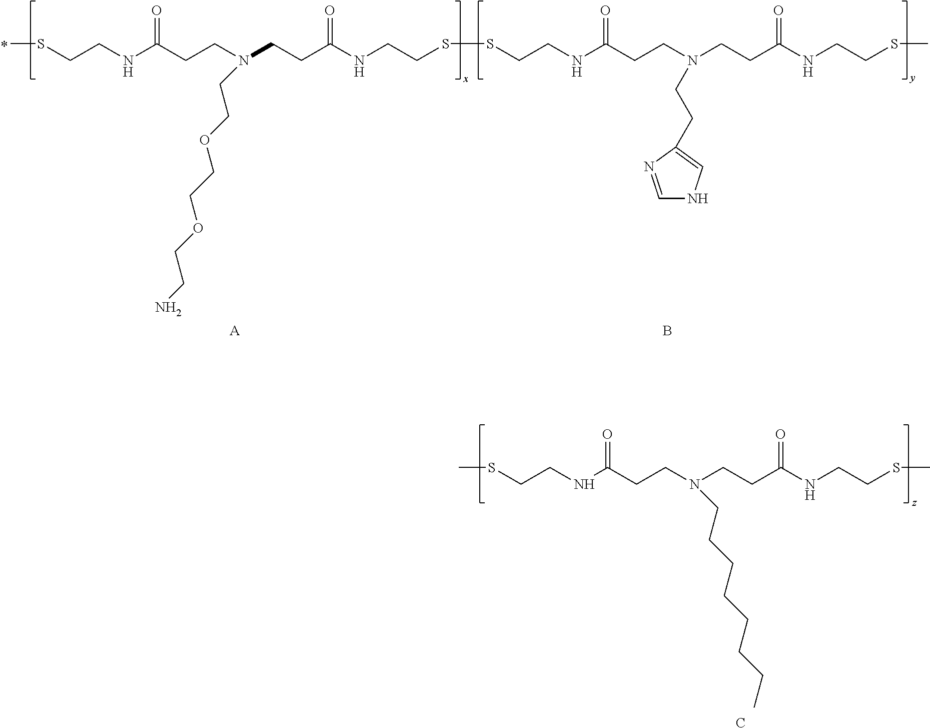

wherein x, y and z are between 0 to 50 and individual polymers in Table 6 include substituents A, B and C in the identified % ratios.

TABLE 6

| A | B | C |
|---|---|---|
| 40 | 30 | 30 |
| 30 | 20 | 50 |
| 70 | 0 | 30 |
| 50 | 0 | 50 |
| 50 | 10 | 40 |

### Polymer 7

Polymers of the instant invention include:

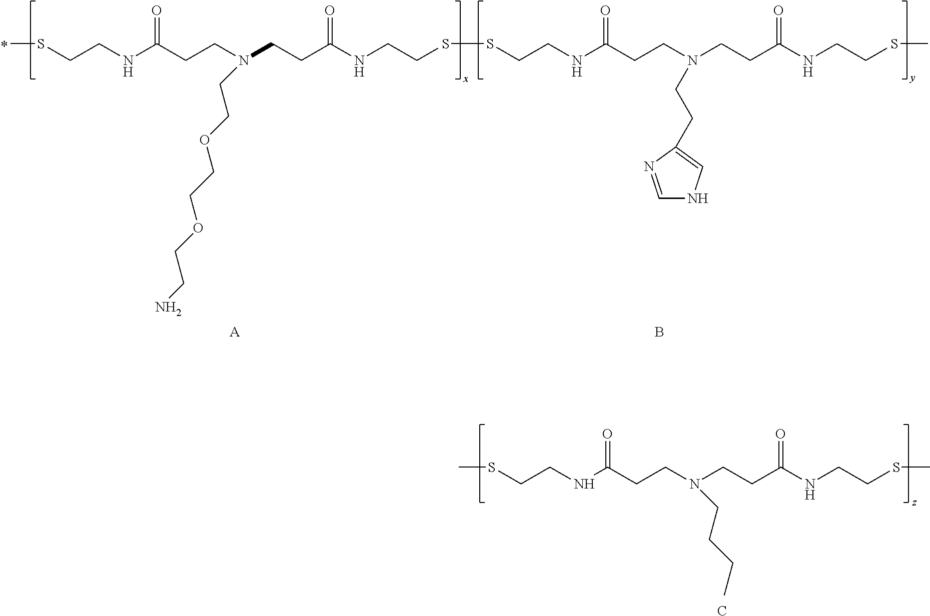

wherein x, y and z are between 0 to 50 and individual polymers in Table 7 include substituents A, B and C in the identified % ratios.

TABLE 7

| A | B | C |
|---|---|---|
| 70 | 0 | 30 |
| 50 | 0 | 50 |
| 50 | 10 | 40 |

### Polymer 8

Polymers of the instant invention include:

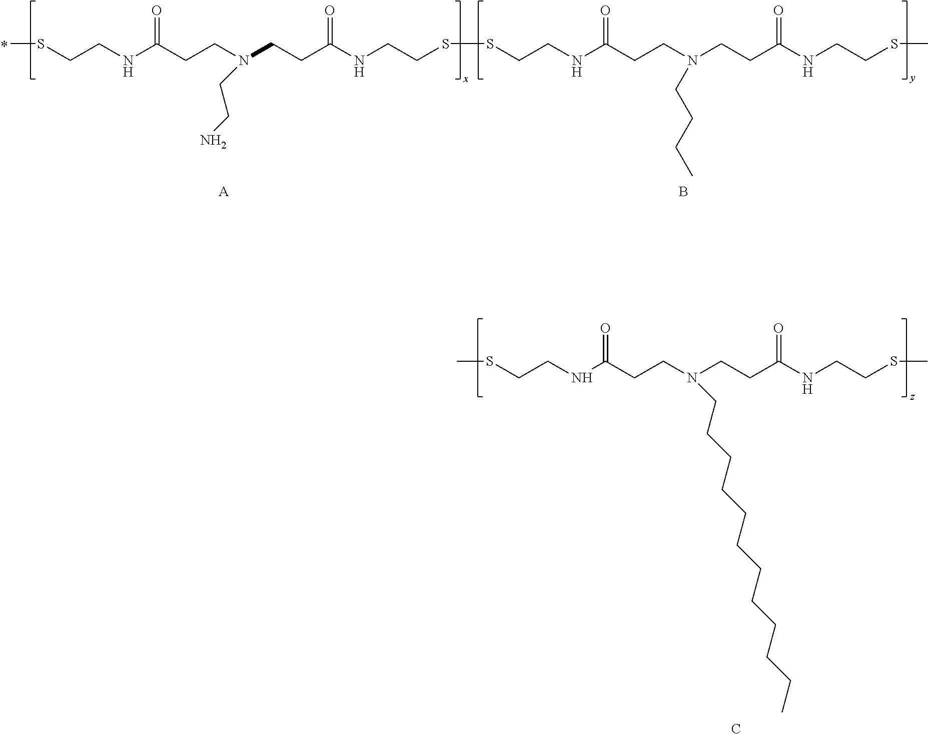

wherein x, y and z are between 0 to 50 and individual polymers in Table 8 include substituents A, B and C in the identified % ratios.

TABLE 8

| A | B | C |
|---|---|---|
| 75 | 20 | 5 |
| 80 | 20 | 0 |
| 80 | 0 | 20 |

### Polymer 9

Polymers of the instant invention include:

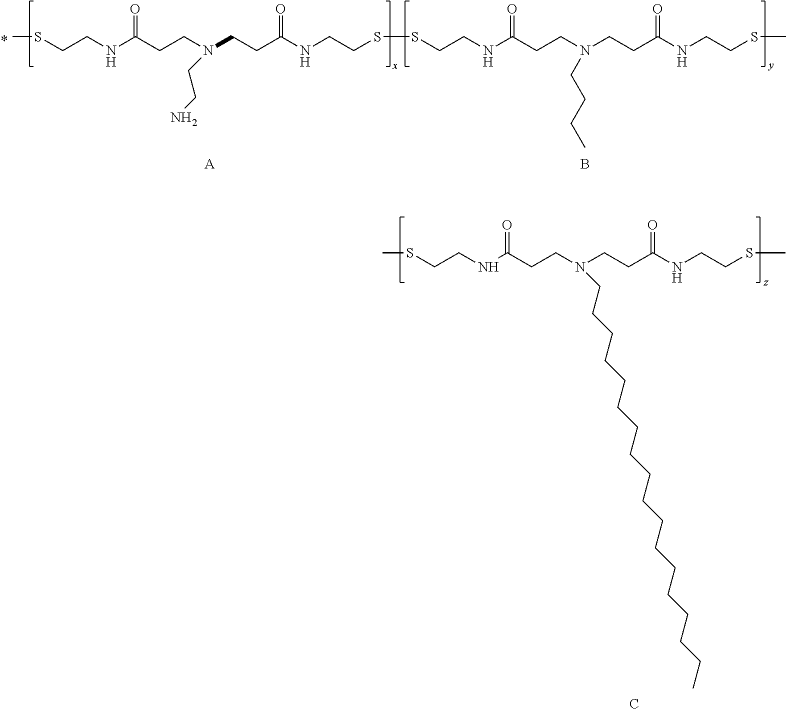

wherein x, y and z are between 0 to 50 and individual polymers in Table 9 include substituents A, B and C in the identified % ratios.

TABLE 9

| A | B | C |
|---|---|---|
| 75 | 20 | 5 |
| 80 | 0 | 20 |

### Polymer 10

Polymers of the instant invention include:

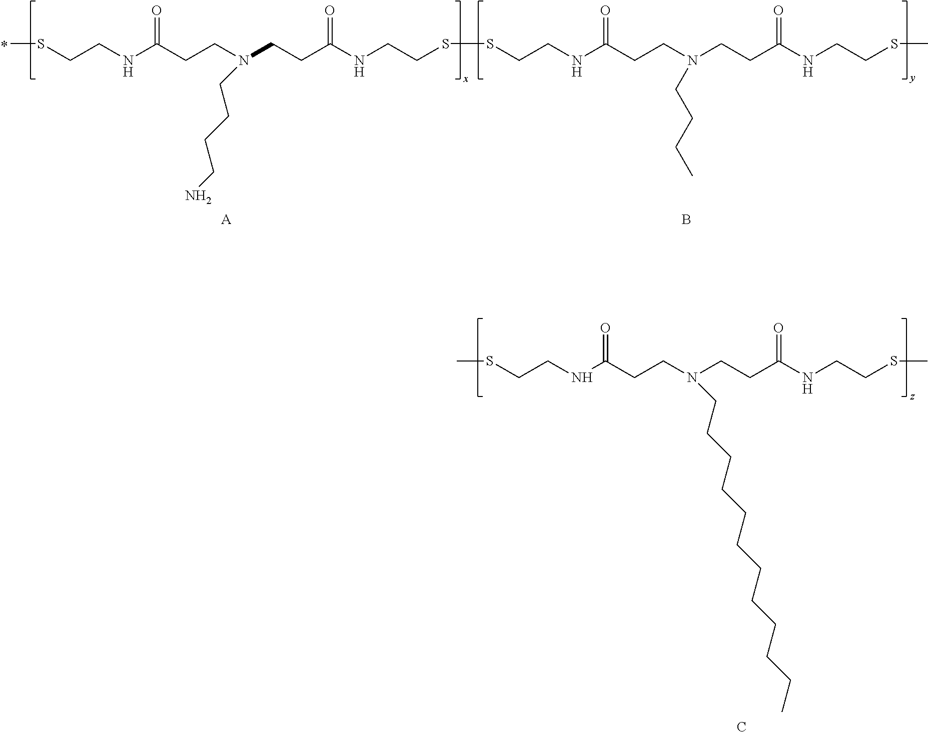

wherein x, y and z are between 0 to 50 and individual polymers in Table 10 include substituents A, B and C in the identified % ratios.

TABLE 10

| A | B | C |
|---|---|---|
| 75 | 20 | 5 |
| 80 | 20 | 0 |
| 80 | 0 | 20 |

### Polymer 11

Polymers of the instant invention include:

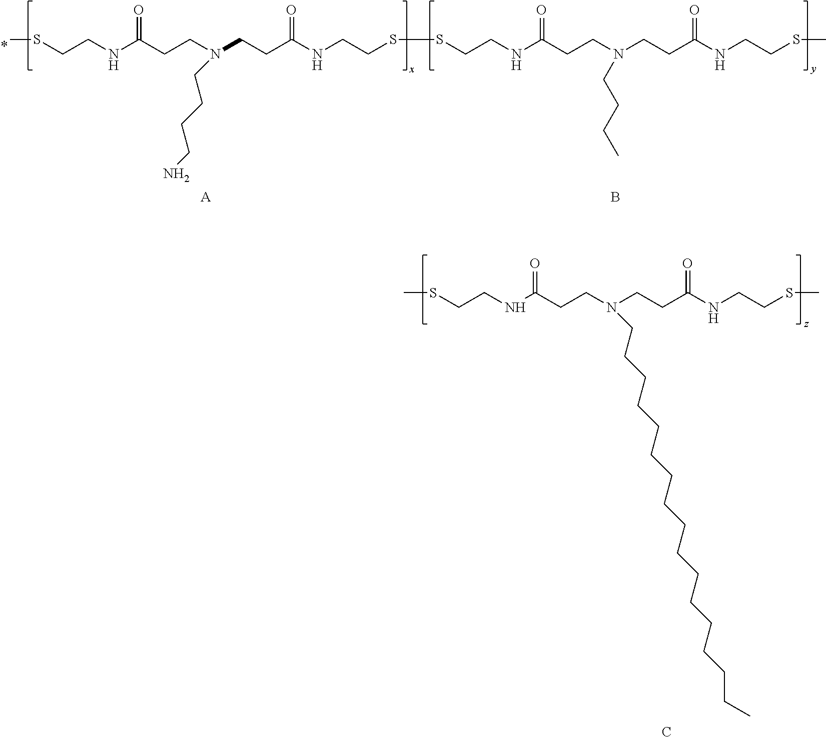

wherein x, y and z are between 0 to 50 and individual polymers in Table 11 include substituents A, B and C in the identified % ratios.

TABLE 11

| A | B | C |
|---|---|---|
| 75 | 20 | 5 |
| 80 | 0 | 20 |

### Polymer 12

Polymers of the instant invention include:

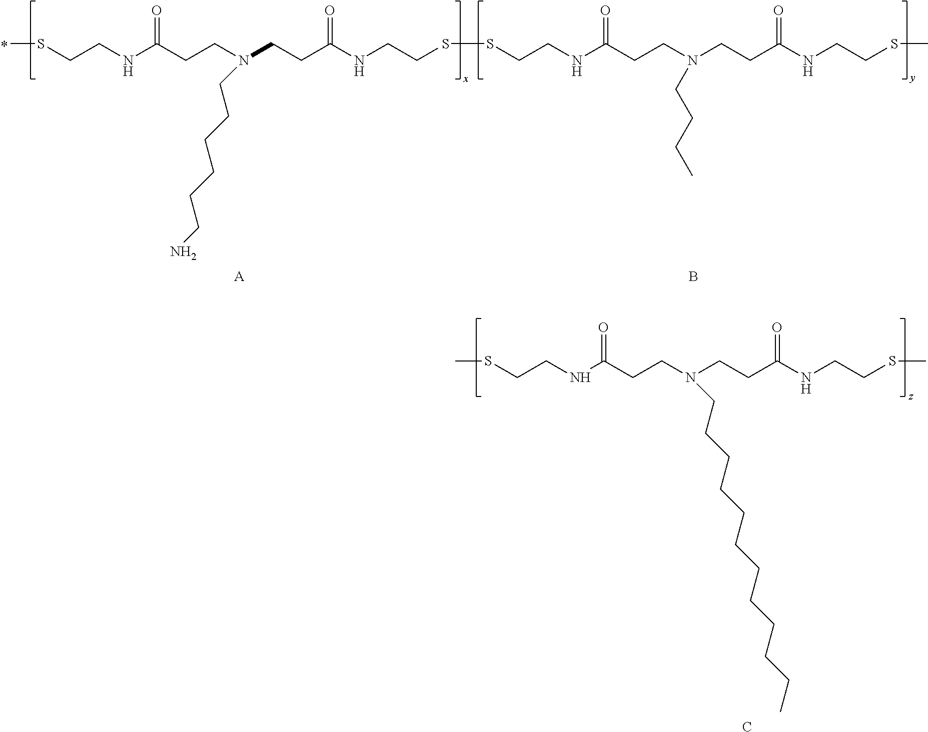

wherein x, y and z are between 0 to 50 and individual polymers in Table 12 include substituents A, B and C in the identified % ratios.

TABLE 12

| A | B | C |
|---|---|---|
| 75 | 20 | 5 |
| 80 | 20 | 0 |
| 80 | 0 | 20 |

### Polymer 13

Polymers of the instant invention include:

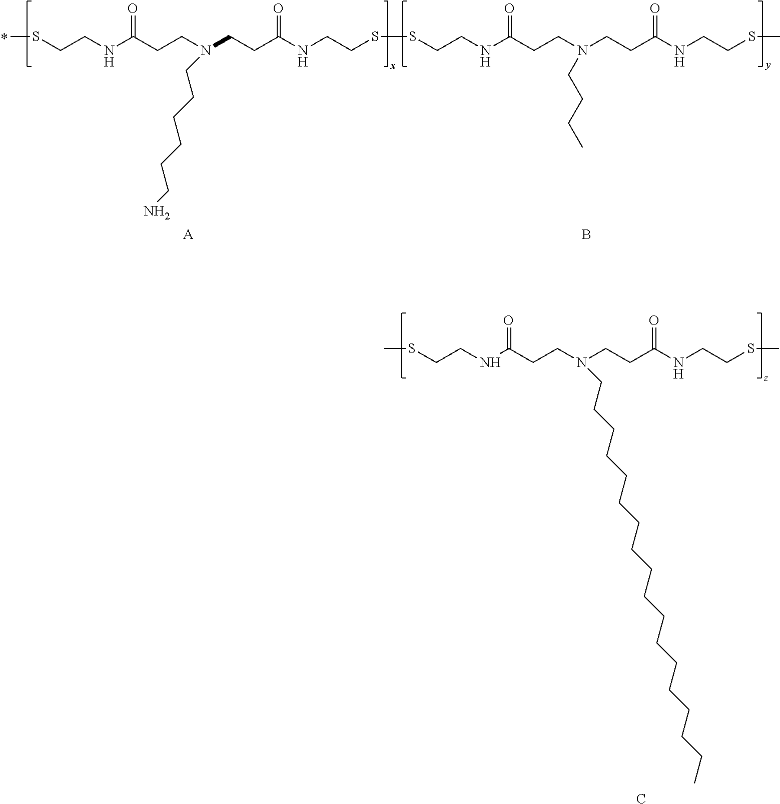

wherein x, y and z are between 0 to 50 and individual polymers in Table 13 include substituents A, B and C in the identified % ratios.

TABLE 13

| A | B | C |
|---|---|---|
| 75 | 20 | 5 |
| 80 | 0 | 20 |

### Polymer 14

Polymers of the instant invention include:

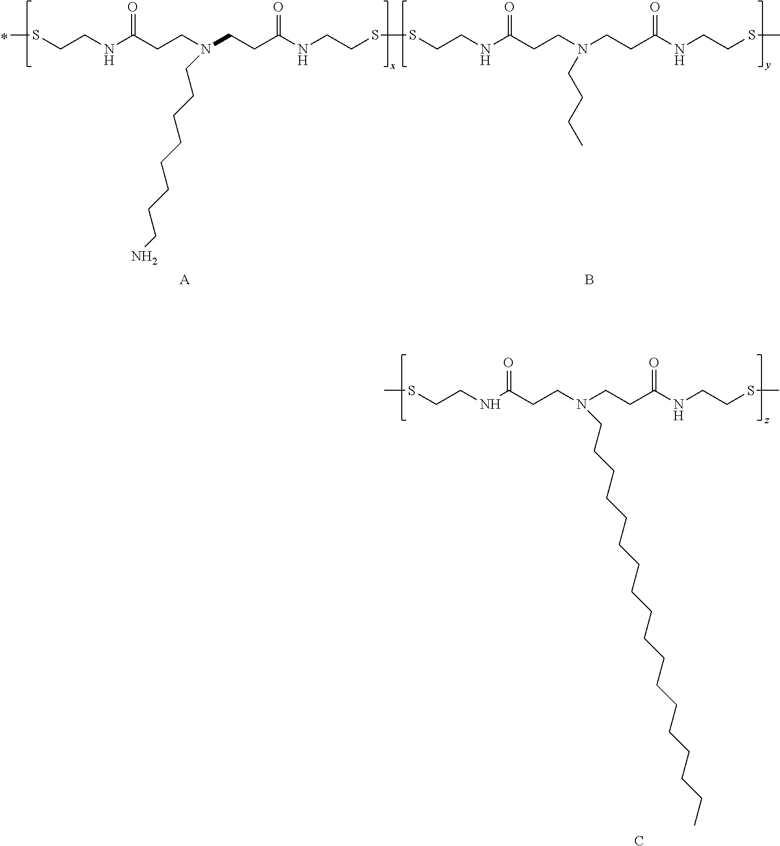

wherein x, y and z are between 0 to 50 and individual polymers in Table 14 include substituents A, 13 and C in the identified % ratios.

TABLE 14

| A | B | C |
|---|---|---|
| 75 | 20 | 5 |
| 80 | 0 | 20 |

### Polymer 15

Polymers of the instant invention include:

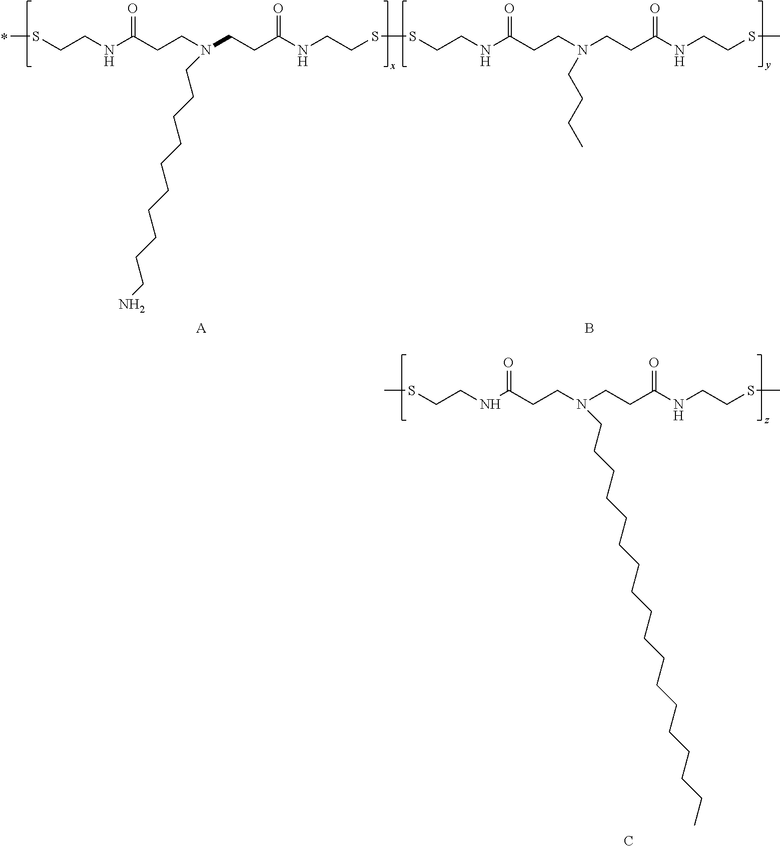

wherein x, y and z are between 0 to 50 and individual polymers in Table 15 include substituents A, B and C in the identified % ratios.

TABLE 15

| A | B | C |
|---|---|---|
| 75 | 20 | 5 |
| 80 | 0 | 20 |

### Conjugation (Scheme 3)

The polymers of Formula Z and the specific examples shown above were synthesized for use in the following conjugation steps to ultimately create the polyconjugates of the instant invention. The polymers of Formula Z and the specific examples disclosed are useful in the preparation of polyconjugates which are, in turn, useful for the delivery of nucleic acids, specifically the delivery of siRNA. Other methods for the synthesis of polyconjugates are described in WO2008/022309.

### Step 1: Activation of Polymer

About 2.6 mg of polymer in a 4 mL vial is added with ˜87 μL of 5 mM TAPS, pH 9 buffer and stirred until the polymer is dissolved. To this solution was added 3.9 μl, of (4-succinimidyloxycarbonyl-<-methyl-<-[2-pyridyldithio]toluene) solution (1 mg/100 μl in DMSO) corresponding to 1.5 wt % with respect to the polymer weight.

Step 2: Activation of siRNA

siRNA (1 g, 0.0714 mmol) is dissolved in 0.1M sodium bicarbonate buffer (20 ml, 50 mg/mL) in a vial with magnetic stir bar and cooled to 0-5° C. in an ice water bath. In a separate vial N-Succinimidyl-5-acetylthioacetate (SATA) (83 mg, 0.357 mmol, 5 equivalents) is dissolved in 0.78 ml DMSO. The SATA solution is added over 1 min and the clear, colorless reaction mixture stirred at 0-5° C. for 2 h. After 2 h, the reaction mixture is sampled and analyzed by HPLC or HPLC for completion of the conjugation. If >5% siRNA remains unreacted, another charge of SATA in DMSO (2.0 equivalents) is added and the reaction aged at 0-5° C. for completion of the SATA conjugation (confirmation by HPLC or HPLC). When there is <5% unreacted siRNA remaining by HPLC or HPLC, the reaction mixture is purified by TFF dialysis using water (˜2 L) or PD10 column to remove any remaining SATA/succinimides. The recovered purified solution is lyophilized to a white fluffy solid. The recovery is typically around 95% and the purity is greater than 70% by HPLC.

### Step 3: Polymer-siRNA Conjugation

The activated polymer is diluted with additional 5 mM TAPS 5% glucose buffer pH 9 resulting in a final polymer concentration of ˜1.9 mg/mL. About 0.66 mg of siRNA is added to the activated polymer solution and stirred at room temperature overnight and proceeded to final masking step.

### Step 4: Masking of Polymer Conjugate

To the polymer-siRNA conjugate add 1.15 mg of TAPS free base. In a separate vial, 0.4 mg of carboxydimethylmaleic anhydride-polyethyleneglycol (CDM-PEG) is weighed and 0.775 mg of carboxydimethylmaleic anhydride-N-acetylgalactosamine (CDM-NAG) is added to this. The siRNA-polymer conjugate solution is then transferred into this vial containing CDM-PEG and CDM-NAG and stirred for 1 hr at room temperature.

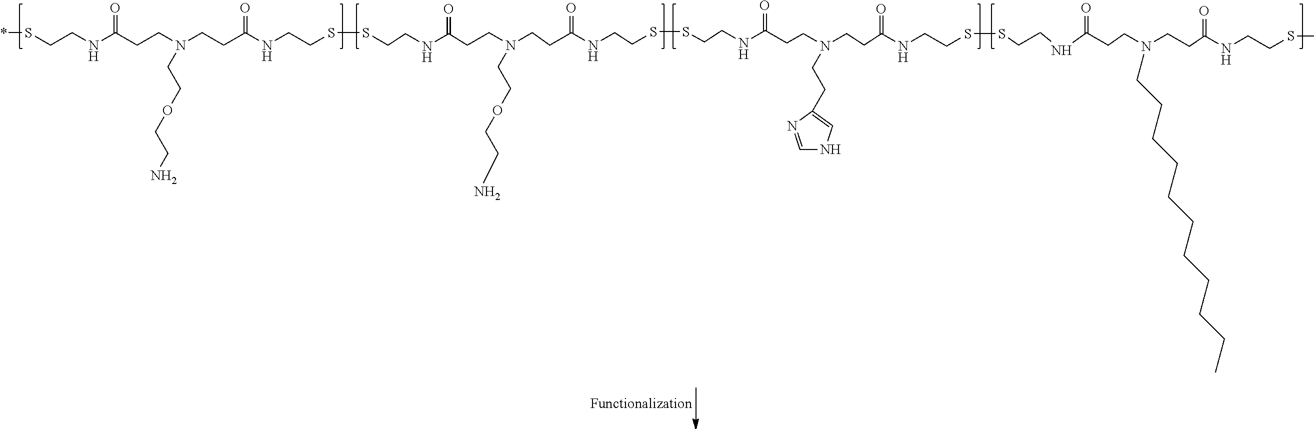

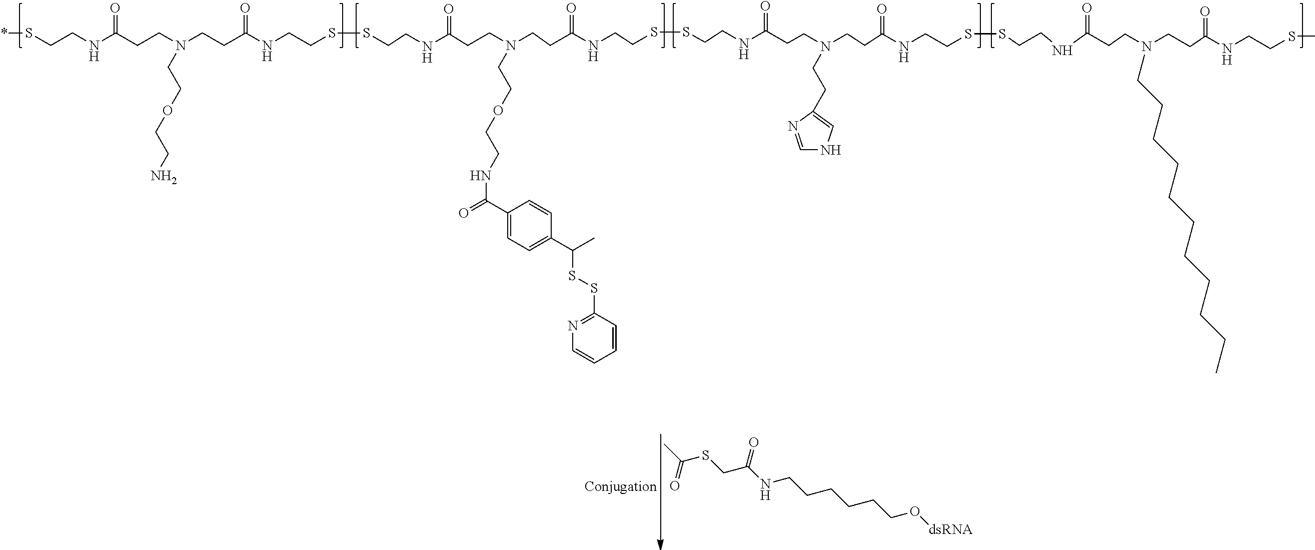

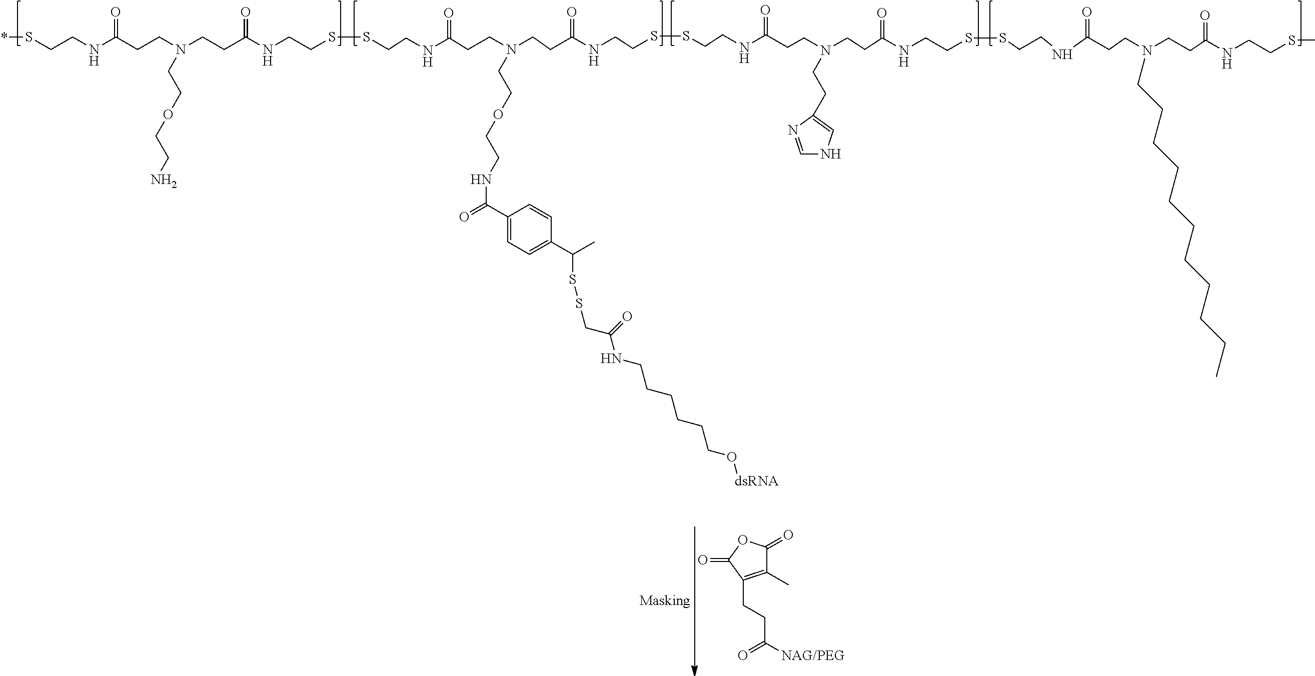

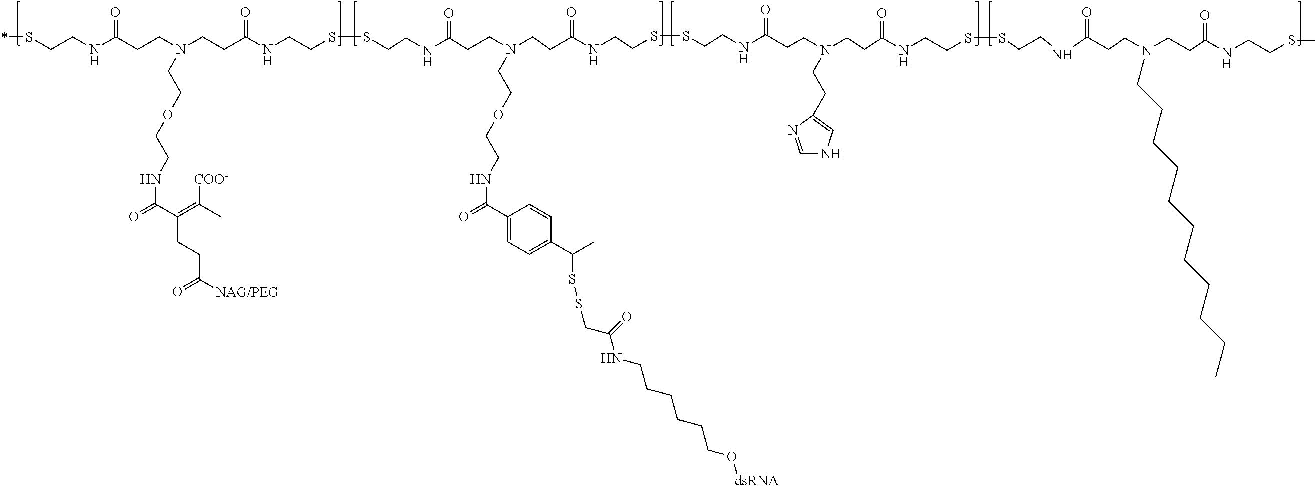

### Example 1

### RBC Hemolysis Assay:

Human blood was collected in 10 ml EDTA Vacutainer tubes. A small aliquot was assessed for evidence of hemolysis by centrifugation at 15000 RCF for 2 min and non-hemolyzed samples were carried forward into the assay. Red blood cells (RBCs) were washed three times in either 150 mM NaCl/20 mM MES, pH 5.4, or 150 mM NaCl/20 mM HEPES, pH 7.5 by centrifuging at 1700×g for 3 min and resuspending in the same buffer to yield the initial volume. RBCs were then diluted in appropriate pH buffer to yield 10⁸ cells in suspension. A 10× stock concentration of each test agent (Polymer 1, Polymer 2, Polymer 3 and Polymer 4) was prepared and a 10 point, 2-fold dilution was performed in appropriate pH buffers. The diluted test agents were added to the RBCs in appropriate pH buffers in Costar 3368 flat-bottom 96 well plates. Solutions were mixed 6 to 8 times and the microtiter plate was covered with a low evaporation lid and incubated in a 37° C. warm room or incubator for 30 minutes to induce hemolysis. The plate was then centrifuged at 1700×g for 5 min and 150 μl supernatants were transferred to a Costar 3632 clear bottom 96 well plate. Hemoglobin absorbance was read at 541 nM using a Tecan Satire plate reader and percent hemolysis was calculated assuming 100% lysis to be measured by the hemoglobin released by RBCs in 1% Triton X-100.

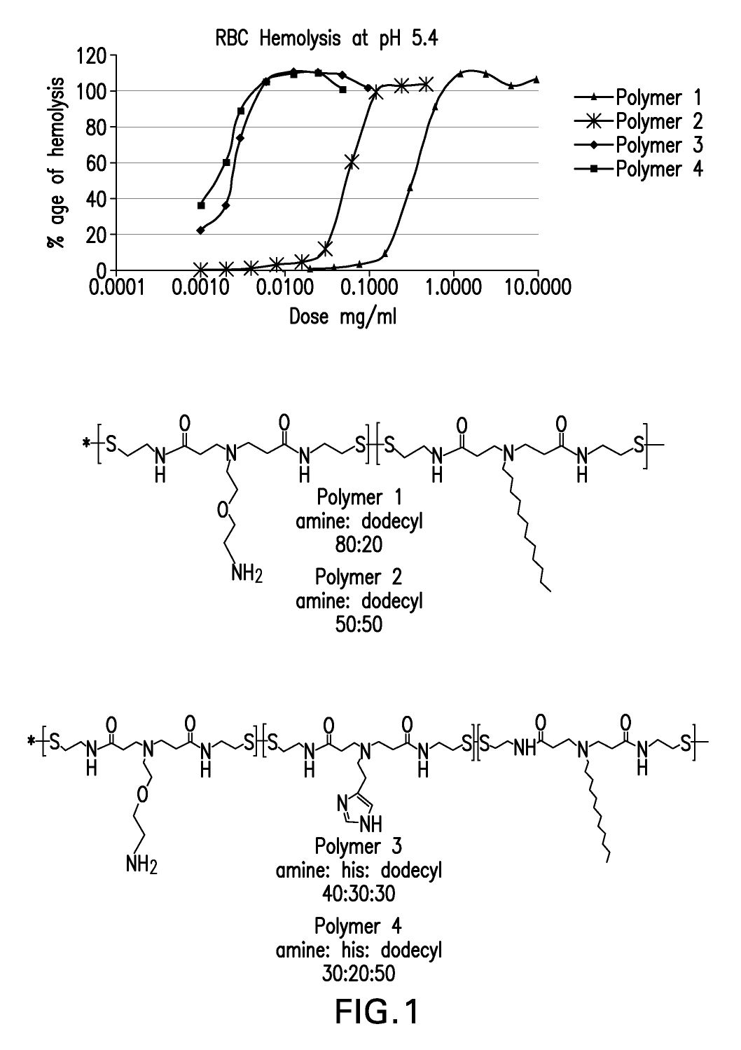

As shown in FIG. 1, the data demonstrate that the polymers are lytic at endosomal pH 5.4. As the ratio of hydrophobic group (dodecyl) increases in the polymer from 20% to 50% the lytic activity increases. Incorporation of histidine in the polymers further enhanced the lytic activity of polymer.

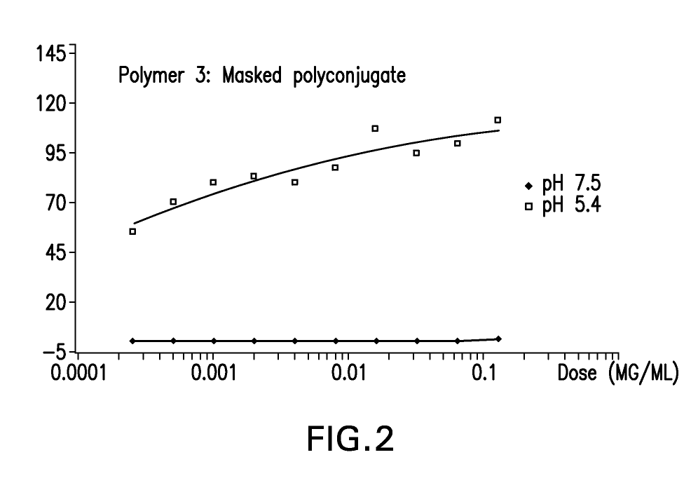

As shown in FIG. 2, the data demonstrates that in extracellular environment at pH 7.5, the polymer is masked with CDM and do not have any lytic activity. At endosomal pH 5.4, after the demasking of CDM, the polymer retains its lytic properties.

### Example 2

### HepG2 Gene Silencing and Toxicity Data:

HepG2 cells were plated in 96-well microtiter plates at 6000 cells/well and incubated overnight at 37° C. to allow cell adherence. 10× stock of PCs (polyconjugates) were prepared in media and 20 μl 10× PC was added to 180 μl media already in wells resulting in 1× final treatment and a 300-0 nM 10-point half log titration, based on siRNA concentration. Cells were incubated with PCs in 37 degrees CO₂ incubator for 24-72 h. MTS Toxicity Assay was performed on 24 h-72 h treated cells and cytotoxicity was assessed by CellTiter 96 Aqueous One Solution Cell Proliferation Assay (Promega #G3581, Madison, Wis.). 40 μl MTS Solution was added, incubated in 37 degrees CO₂ incubator 1 hour, absorbance at 490 nm was read on Tecan Safire. Cells were then washed 3× in PBS and 150 μl/well bDNA DLM Lysis Buffer (Panomics “Quantigene” 1.0 bDNA kit #QG0002, Fremont, Calif.) was added. Plate was then incubated at 37 degrees in Warm Room 30 min. Lysates were removed and frozen at −70 degrees C. overnight. The next day, all cell lysates were thawed at RT and 20 μl of each lysate was removed and used for determination of total protein using Micro BCA Protein Assay kit (Pierce #23235, through Thermo Scientific, Rockford, Ill.). Absorbance was measured on Tecan Satire: Wavelength=562 nM, Plate=Costar96ft, Number of Reads=100, Time between Reads=5. 50 μl each lysate was also used to determine mRNA expression levels in cells treated with SSB siRNA.

ApoB mRNA knockdown was determined using Quantigene 1.0 bDNA Assay (Panomics 4 QG0002 Lot #51 CW36, Fremont, Calif.), a kit designed to quantitate RNA using a set of target-specific oligonucleotide probes.

Zimmerman Apo B siRNA was utilized in the experiments. Zimmermann et al., (2006) Nature, 441(7089):111-114 or doi:10.1038/nature04688 (see supplementary information). Panomics Quantigene bDNA Kit #QG0002-protocol for 96 well plate:

### Day 1

Make diluted lysis mixture (DLM) by mixing 1 volume of lysis mixture with 2 volumes of Nuclease Free water (Ambion cat #AM9930). Aspirate (PBS) from plate. Add 150 μl DLM to each well and mix. (Include Column 1 as Buffer Alone Background). Incubate at 37° C. for 30 minutes. (After heating, Lysates can be placed in the −70° C. freezer until analysis is performed. If lysates are frozen, thaw at Room Temperature and incubate at 37° C. for 30 minutes and mix well before adding the samples to the capture plate.) Bring all reagents to Room Temperature before use, including the capture plates. Dilute CE, LE and BL probe set components: 0.1 μl/well each into DLM. Add (100-X) μl diluted probe set/well. Add (X) μl cell lysate/well. Cover with foil plate sealer. Incubate at 53° C. for 16-20 hrs. Note: If assay contains multiple plates, perform steps 7, 8, 9 on 2-3 plates at a time and place at 53° C. before going on to next 2-3 plates.

### Day 2

Bring Amplifier, Label Probe and Substrate to Room Temperature. Vortex and briefly centrifuge the tubes of Amplifier and Label Probe to bring the contents to the bottom of the tube. Prepare Wash buffer: add 3 ml Component 1 and 5 ml Component 2 to 1 L distilled water. (Wash Buffer is stable at Room Temperature for up to 6 months)

Prepare as needed: Amplifier Working solution, Label Probe Working Solution, and Substrate

### Working Solution:

Amplifier Working Solution—1:1000 dilution into Amplifier/Label Probe diluent.
Label Probe Working solution—1:1000 dilution into Amplifier/Label Probe diluent.
Substrate Working Solution—1:333 dilution of 10% Lithium Lauryl Sulfate Substrate into Substrate Solution (protect from light).

Add 200 μl/well of wash buffer to overnight hybridization mixture. Repeat washes 3× with 300 μl of Wash Buffer. *Do not let the capture plates stand dry for longer than 5 minutes. Add 100 μl/well of Amplifier Working Solution. Seal plate with clear seal and incubate at 53° C. for 30 minutes. Wash plate 3× with 300 μl of Wash Buffer. Add 100 μl/well of Label Probe Working Solution. Seal plate with clear seal and incubate at 53° C. for 30 minutes. Wash plate 3× with 300 μl of Wash Buffer. Add 100 μl/well Substrate Working Solution. Seal plate with foil seal and incubate at 53° C. for 15 minutes. Let plate stand at Room Temperature for 10 minutes. Read in luminometer with integration time set to 0.2 seconds. bDNA data was normalized to protein and graphed using GraphPad Prism Program using non-linear regression curve fit analysis.

### In Vivo Evaluation of Efficacy

CD1 mice were tail vein injected with the freshly prepared siRNA containing polymer conjugates at a dose of 3 mg/kg in a volume of 0.2 mL, 100 mM TRIS 5% glucose, pH9, vehicle. Forty-eight hours post dose, mice were sacrificed and liver tissue samples were immediately preserved in RNALater (Ambion). Preserved liver tissue was homogenized and total RNA isolated using a Qiagen bead mill and the Qiagen miRNA-Easy RNA isolation kit following the manufacturer's instructions. Liver ApoB mRNA levels were determined by quantitative RT-PCR. Message was amplified from purified RNA utilizing primers against the mouse ApoB mRNA (Applied Biosystems Cat. No. Mm01545156_m1). The PCR reaction was run on an ABI 7500 instrument with a 96-well Fast Block. The ApoB mRNA level is normalized to the housekeeping PPIB mRNA and GAPDH. PPIB and GAPDH mRNA levels were determined by RT-PCR using a commercial probe set (Applied Biosytems Cat. No. Mm00478295_m1 and Mm4352339E_m1). Results are expressed as a ratio of ApoB mRNA/PPIB/GAPDH mRNA. All mRNA data is expressed relative to the vehicle control.

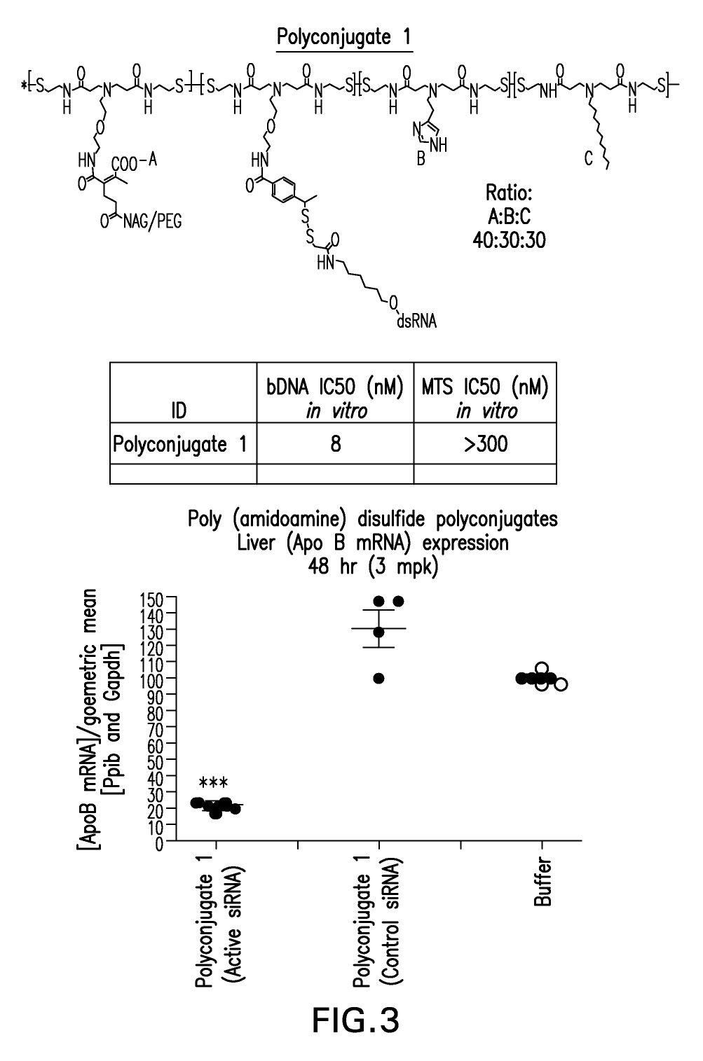

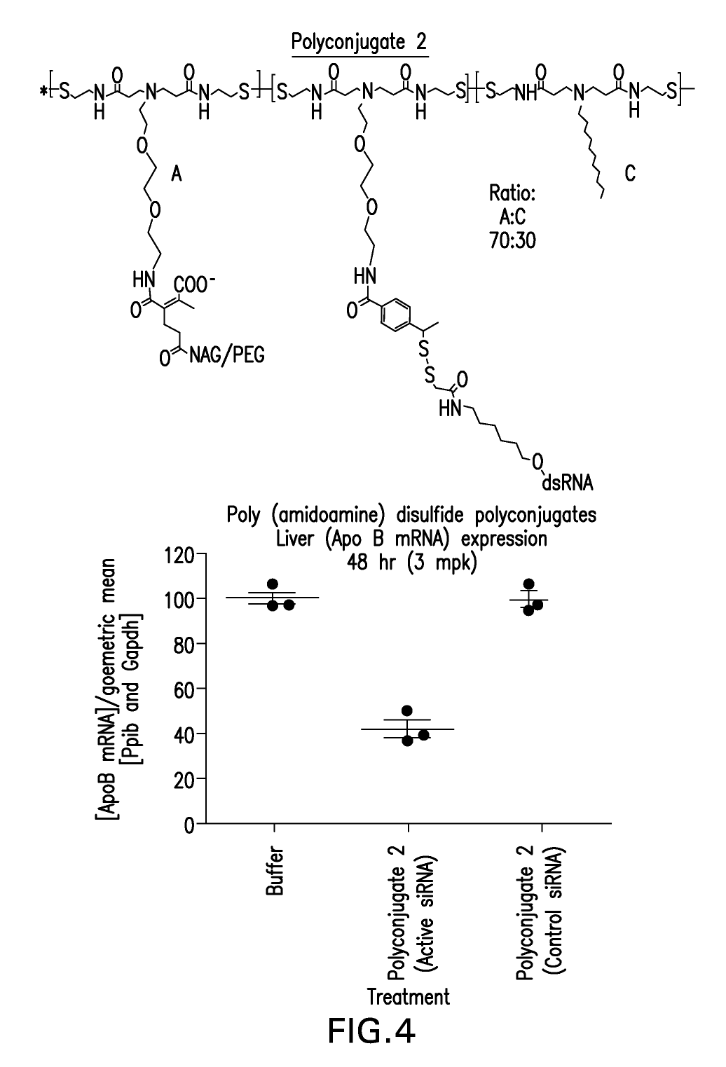

As shown in FIGS. 3 and 4, the data demonstrate that the polyconjugates of the instant invention can deliver siRNA both in vitro and in vivo. In mice, 65-80% knockdown of ApoB was observed with 3 mg/kg dose.

What is claimed is:

1. A polymer according to the Formula Z: 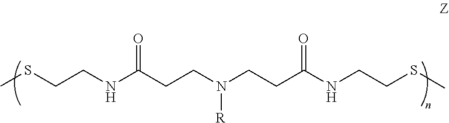
wherein:
n is 2 to 250;
R is independently selected from A, B and C, wherein at least two of A, B and C are present in the polymer and the polymer must include at least one cationic component (A or B) and at least one aliphatic component (B or C);
A is a first cationic component;
B is a second cationic component or a second aliphatic component; and
C is a first aliphatic component;
or stereoisomer thereof.

2. A polymer according to claim 1 of the Formula Z: 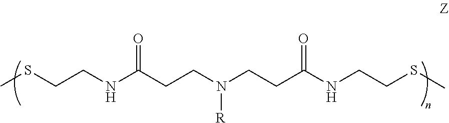
wherein:
n is 2 to 250;
R is independently selected from A, B and C, wherein at least two of A, B and C are present in the polymer and the polymer must include at least one cationic component (A or B) and at least one aliphatic component (B or C);
A is a first cationic component selected from an amine (primary, secondary, tertiary or quaternary), a nitrogen heterocycle, an aldimine, a hydrazide and a hydrazone;
B is a second cationic component selected from an amine (primary, secondary, tertiary or quaternary), a nitrogen heterocycle, an aldimine, a hydrazide and a hydrazone, or a second aliphatic component; and
C is a first aliphatic component;
or stereoisomer thereof.

3. A polymer according to claim 2 of the Formula Z: 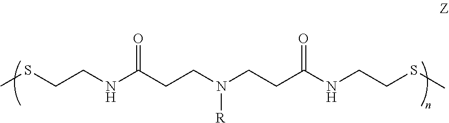
wherein:
n is 2 to 250;
R is independently selected from A, B and C, wherein A, B and C are all present in the polymer;
A is 2-(2-aminoethoxy)ethyl;
B is 2-(1H-imidazol-4-yl)ethyl; and
C is dodecyl;
or a stereoisomer thereof.

4. A polymer according to claim 1 of the Formula Z selected from 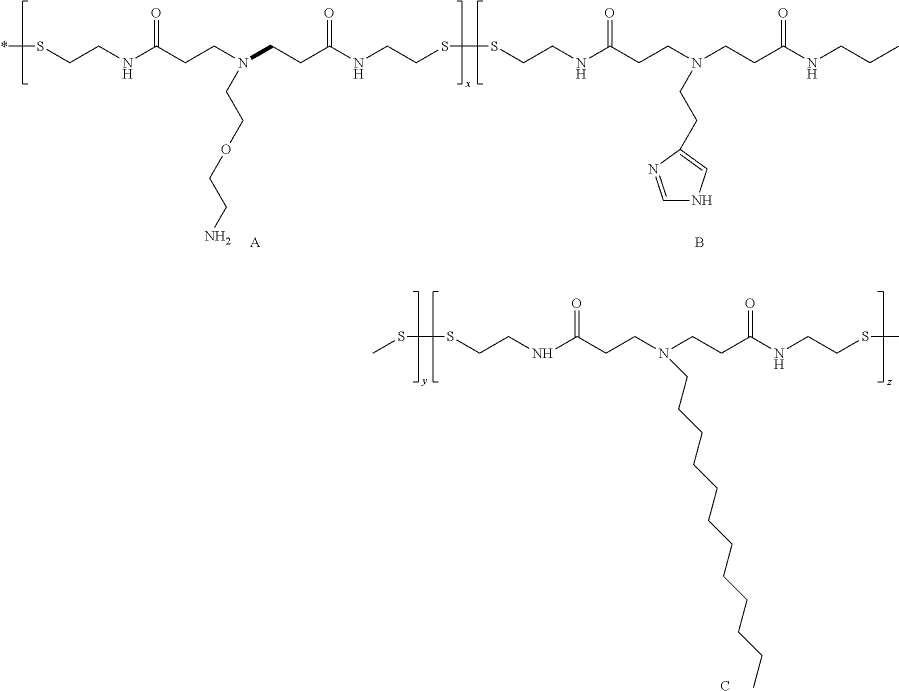
wherein x, y and z are between 0 to 50 and the % ratios of substituents A, B and C are defined in Table 1; 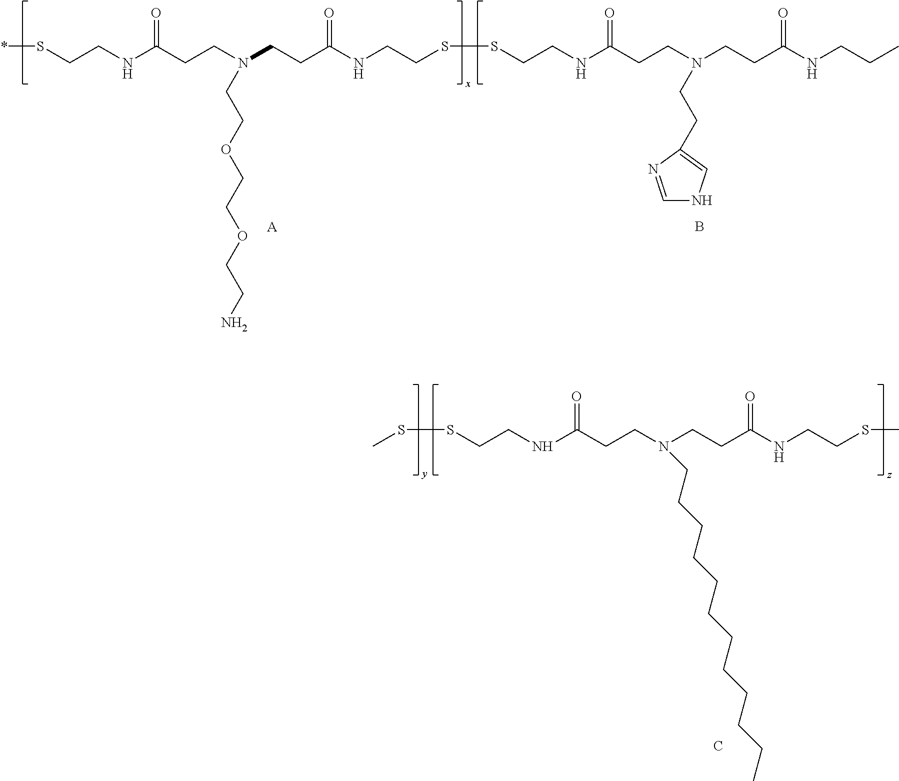
wherein x, y and z are between 0 to 50 and the % ratios of substituents A, B and C are defined in Table 2; 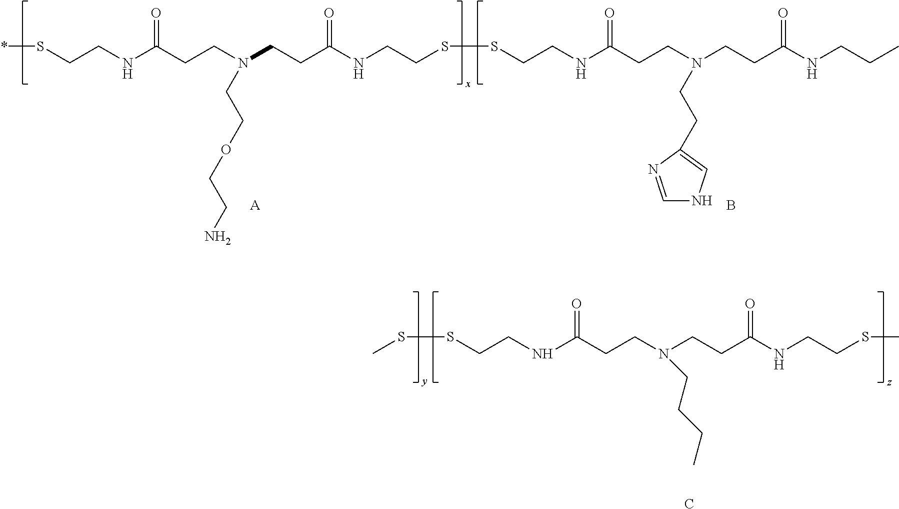
wherein x, y and z are between 0 to 50 and the % ratios of substituents A, B and C are defined in Table 3; 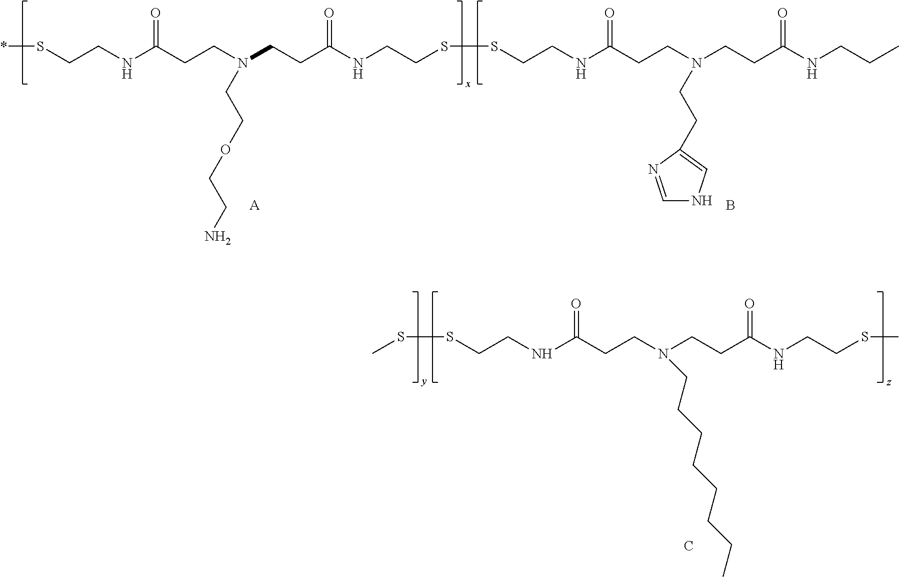
wherein x, y and z are between 0 to 50 and the % ratios of substituents A, B and C are defined in Table 4; 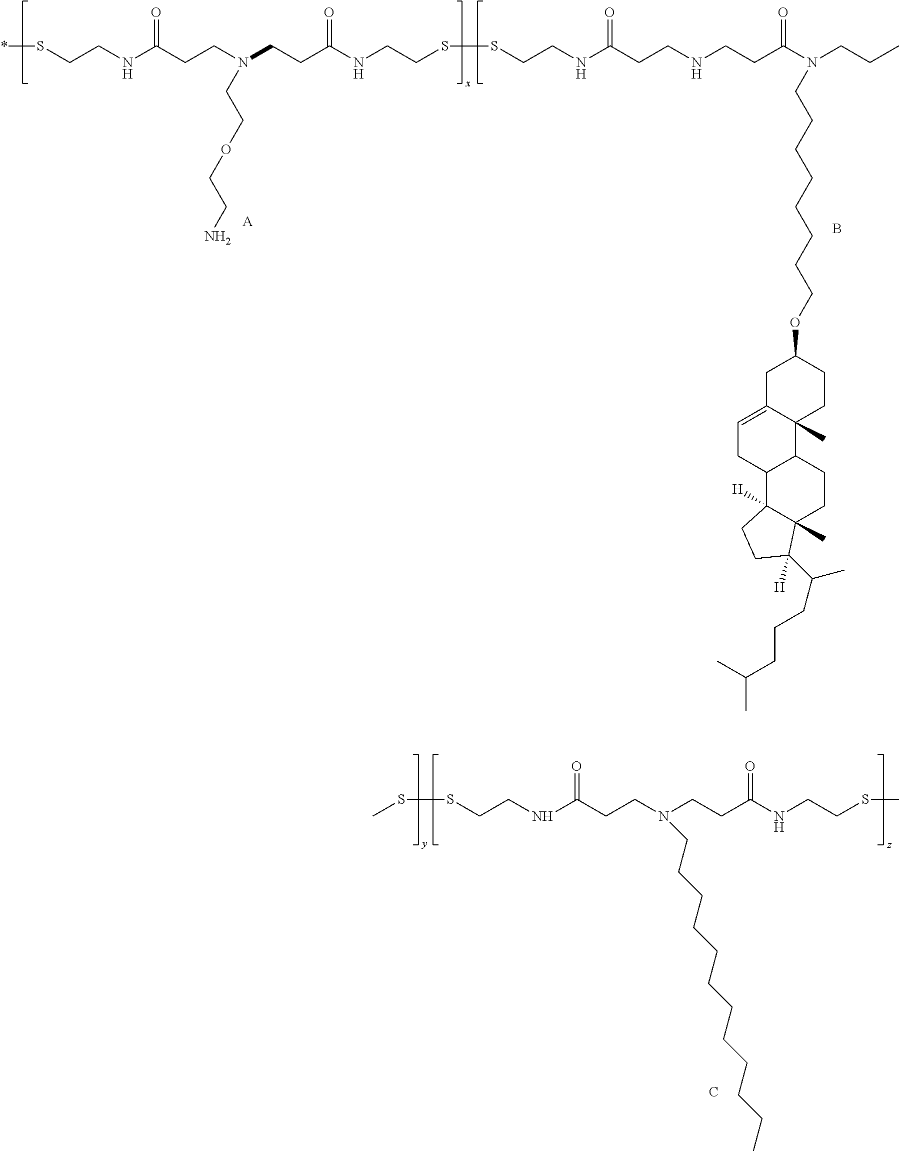
wherein x, y and z are between 0 to 50 and the % ratios of substituents A, B and C are defined in Table 5; 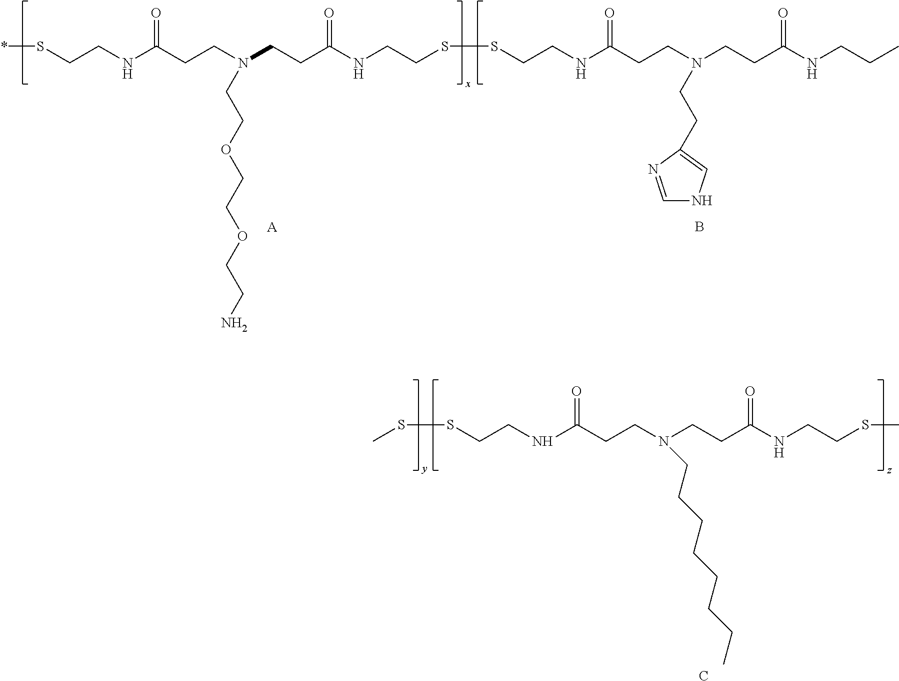
wherein x, y and z are between 0 to 50 and the % ratios of substituents A, B and C are defined in Table 6; 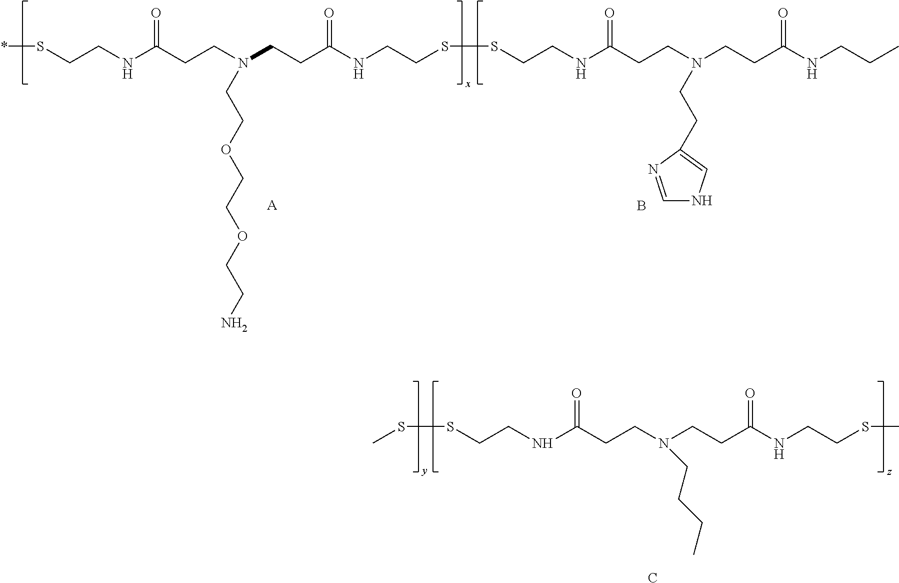
wherein x, y and z are between 0 to 50 and the % ratios of substituents A, B and C are defined in Table 7; 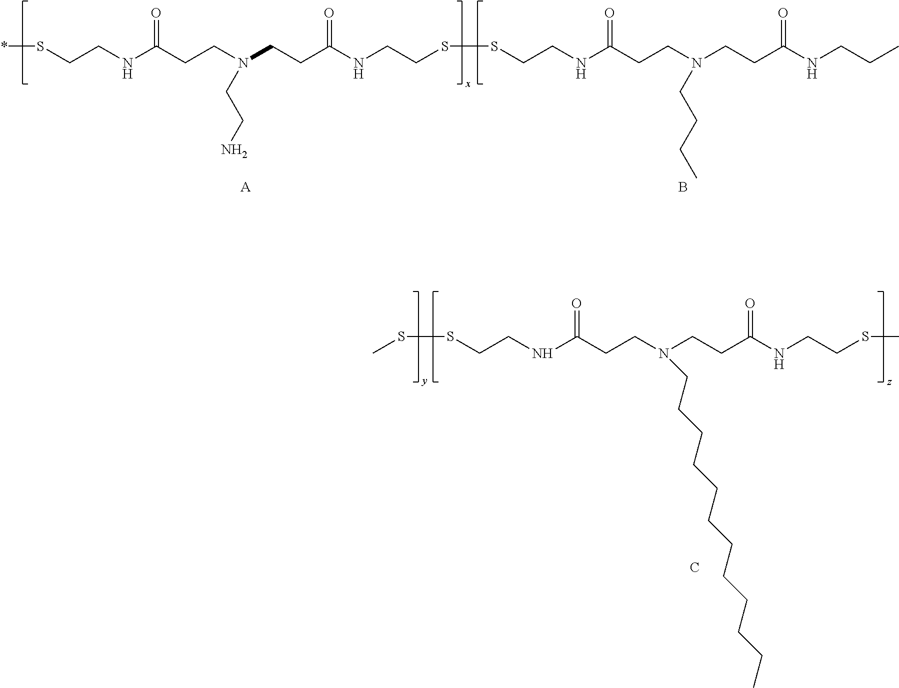
wherein x, y and z are between 0 to 50 and the % ratios of substituents A, B and C are defined in Table 8; 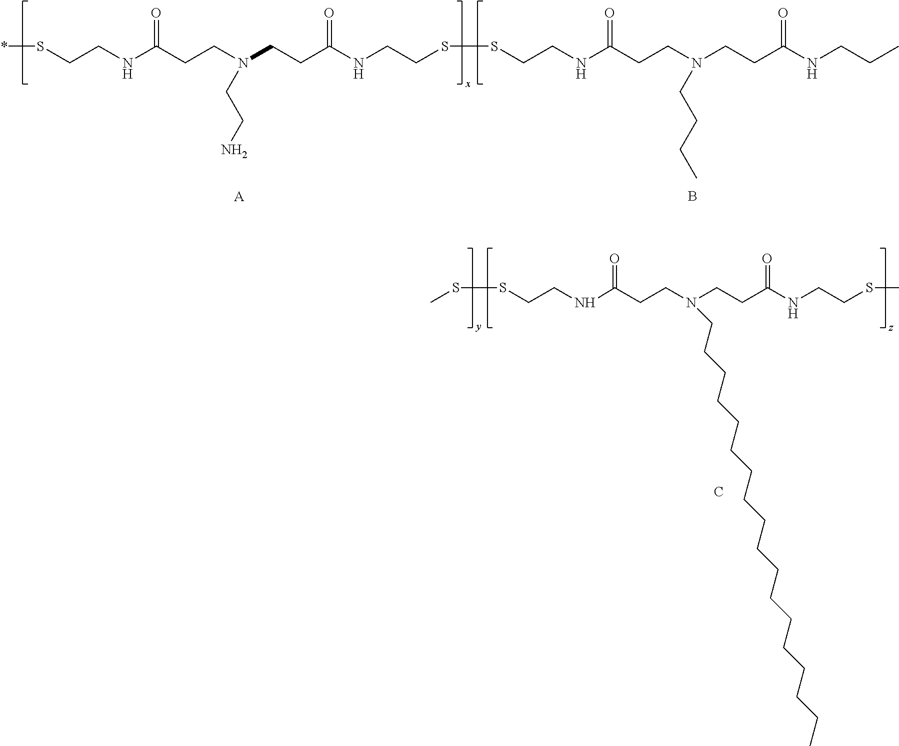
wherein x, y and z are between 0 to 50 and the % ratios of substituents A, B and C are defined in Table 9; 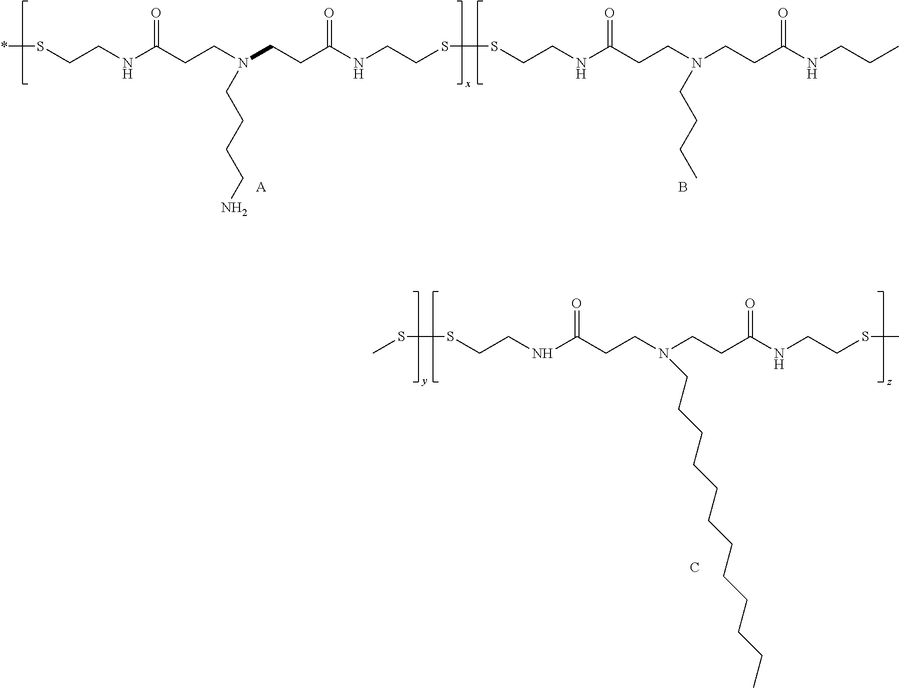
wherein x, y and z are between 0 to 50 and the % ratios of substituents A, B and C are defined in Table 10; 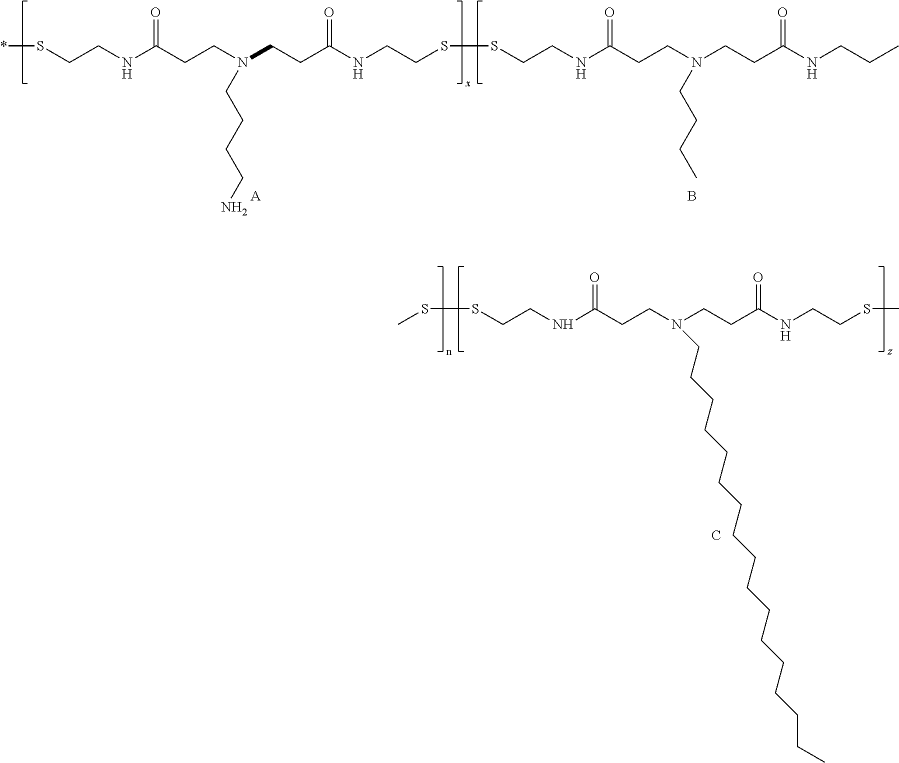
wherein x, y and z are between 0 to 50 and the % ratios of substituents A, B and C are defined in Table 11; 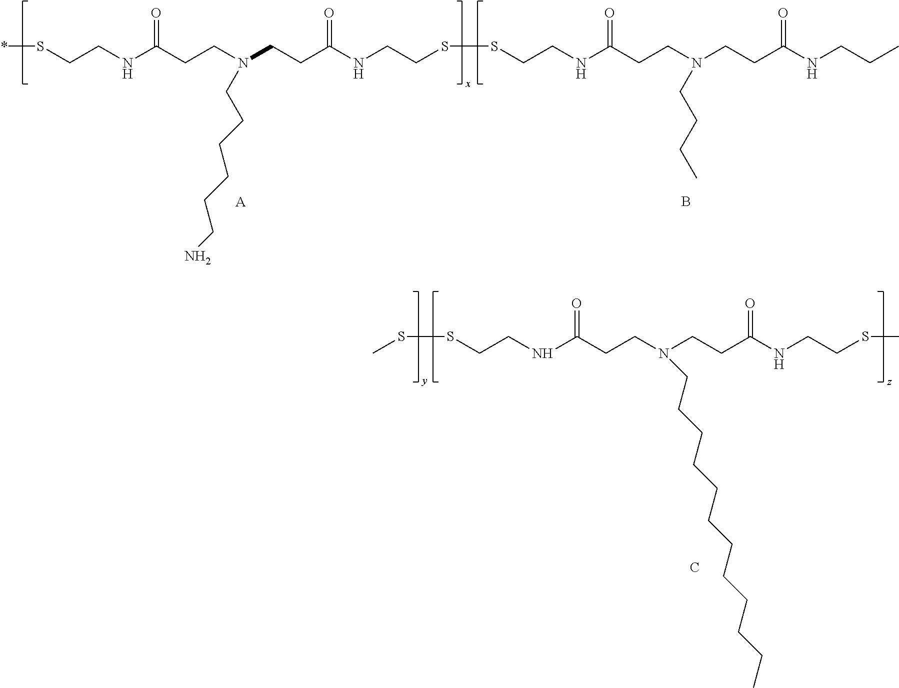
wherein x, y and z are between 0 to 50 and the % ratios of substituents A, B and C are defined in Table 12; 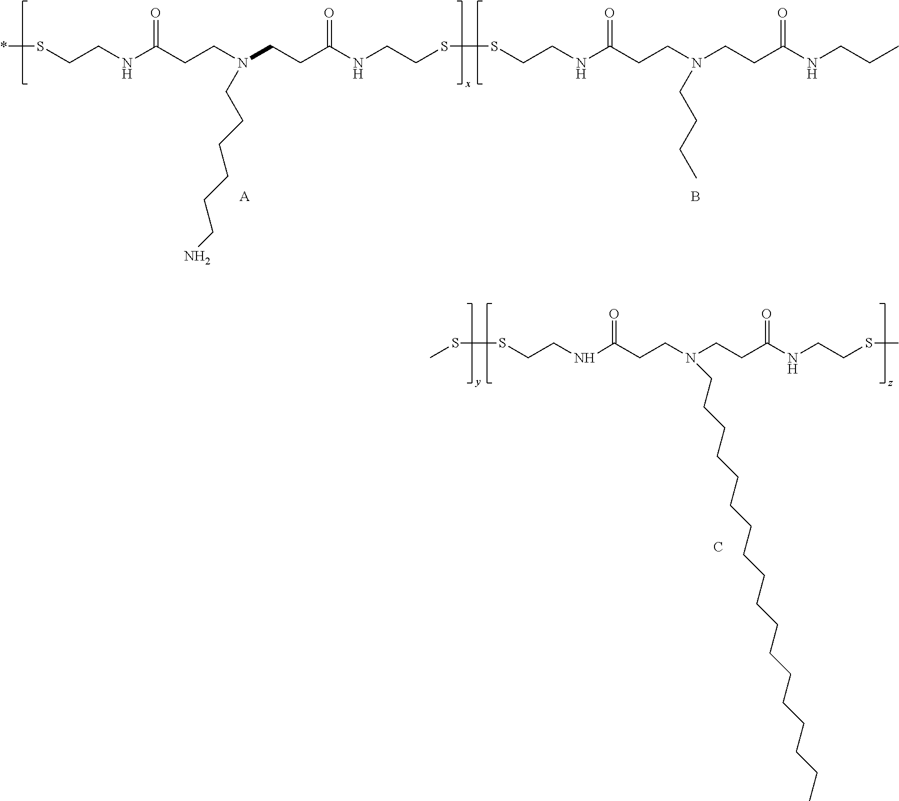
wherein x, y and z are between 0 to 50 and the % ratios of substituents A, B and C are defined in Table 13; 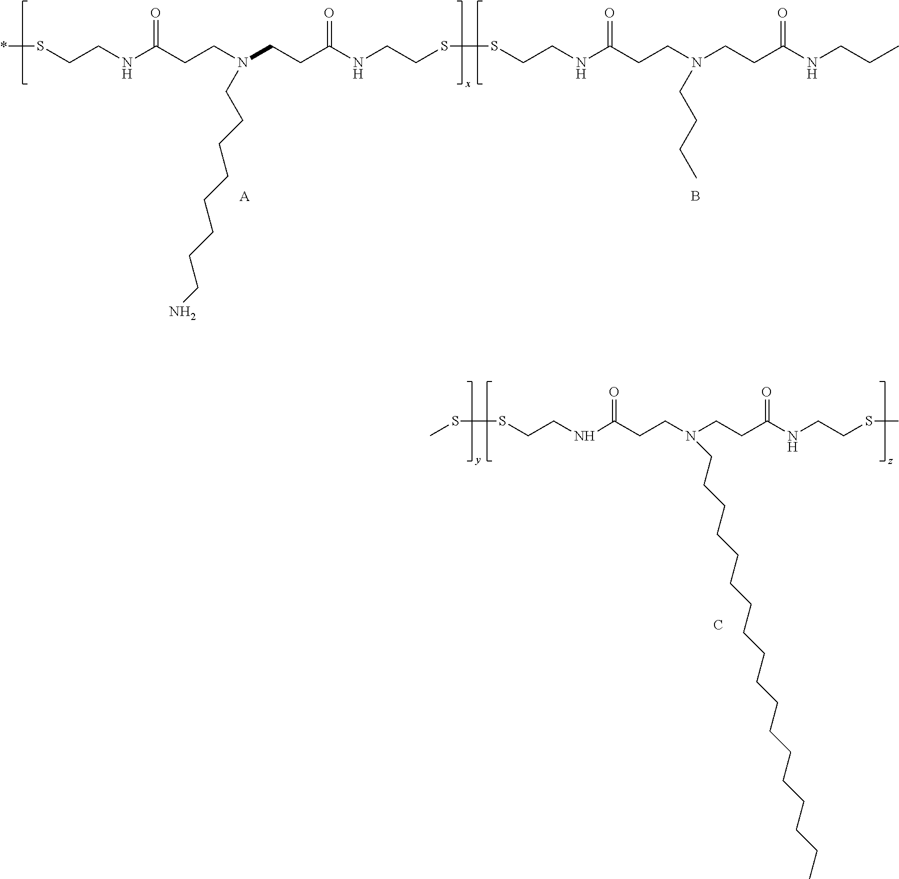
wherein x, y and z are between 0 to 50 and the % ratios of substituents A, B and C are defined in Table 14; and 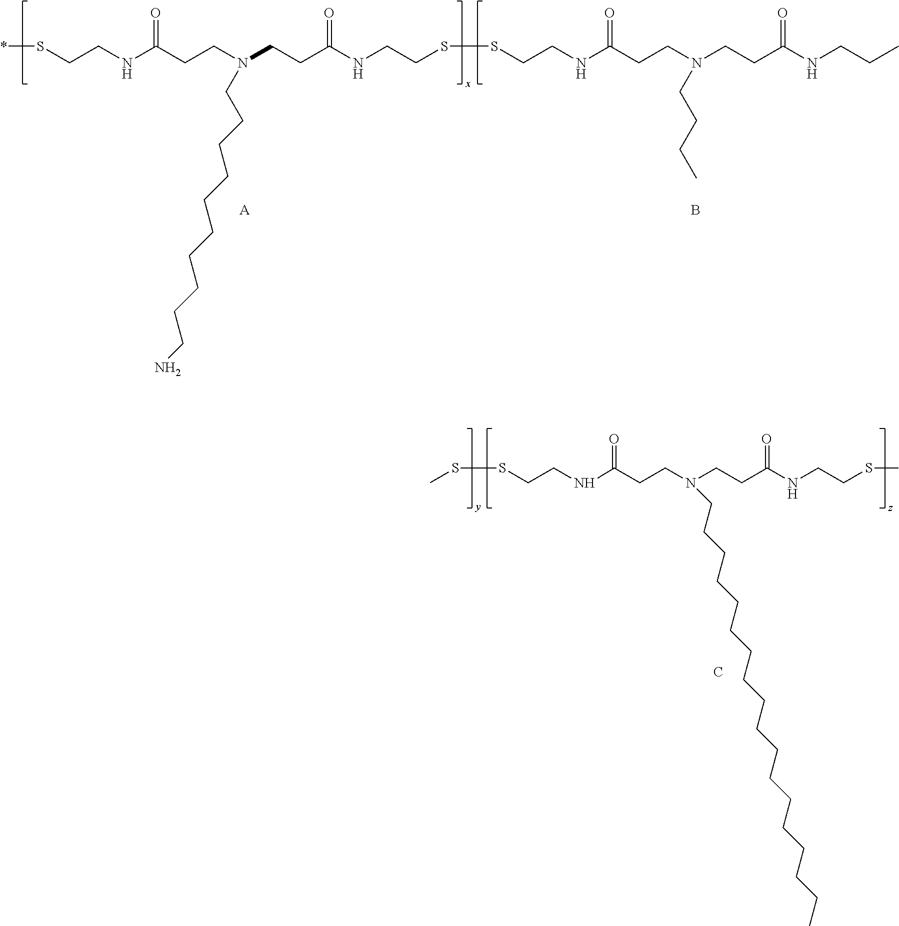
wherein x, y and z are between 0 to 50 and the % ratios of substituents A, B and C are defined in Table 15;
or stereoisomer thereof.

5. The polymer according to claim 1 of the Formula Z wherein substituents A, B and C are all present in the polymer and wherein A, B and C are different from one another.

6. The polymer according to claim 1 of the Formula Z wherein substituent A must comprise at least 50% of the polymer.

7. The polymer according to claim 1 of the Formula Z wherein substituent B must comprise at least 50% of the polymer.

8. The polymer according to claim 1 of the Formula Z wherein substituent C must comprise at least 50% of the polymer.

9. A composition comprising a polymer of Formula Z and an oligonucleotide.

10. A composition according to claim 9 further comprising a masking agent.

11. A composition according to claim 10 further comprising a targeting agent.

12. A method of treating a disease in a patient by administering a composition of claim 9.
